(19) United States
(12) Patent Application Publication
(10) Pub. No.: US 2025/0027020 A1
(43) Pub. Date: Jan. 23, 2025

(54) HYBRID FIXED BED CELL CULTURE SUBSTRATE AND BIOREACTOR

(71) Applicant: CORNING INCORPORATED, CORNING, NY (US)

(72) Inventors: Yujian Sun, Painted Post, NY (US); Yue Zhou, Horseheads, NY (US)

(21) Appl. No.: 18/714,867

(22) PCT Filed: Nov. 17, 2022
(86) PCT No.: PCT/US2022/050202

§ 371 Date: May 30, 2024

Related U.S. Application Data
(60) Provisional application No. 63/284,153, filed on Nov. 30, 2021.

(51) Int. Cl. C12M 1/12; C12M 3/00

(52) U.S. Cl. CPC C12M 25/14; C12M 23/44

(57) ABSTRACT

A cell culture matrix for culturing cells in a fixed bed reactor is provided. The cell culture matrix includes a plurality of substrate layers in a stacked arrangement, with each layer being a substrate material with an ordered and regular array of openings passing through the layer, where the openings are separated by the substrate material, which has a physical structure that is substantially regular and uniform and that is for growing cell thereon. The plurality of substrate layers includes a first substrate material and a second substrate material that is different from the first substrate material in at least one physical dimension. The first substrate material and the second substrate material make up separate layers of the plurality of substrate layers.

This application claims the benefit of priority under 35 U.S.C. § 119 of U.S. Provisional Application Ser. No. 63/284,153 filed on Nov. 30, 2021, the content of which is relied upon and incorporated herein by reference in its entirety.

### FIELD OF THE DISCLOSURE

This disclosure general relates to substrates for culturing cells and fixed bed bioreactors incorporating such substrates. In particular, the present disclosure relates to fixed beds having a mixture of cell culture substrates and bioreactors with such fixed beds having uniform fluid flow characteristics.

### BACKGROUND

In the bioprocessing industry, large-scale cultivation of cells is performed for purposes of the production of hormones, enzymes, antibodies, vaccines, and cell therapies. Cell and gene therapy markets are growing rapidly, with promising treatments moving into clinical trials and quickly toward commercialization. However, one cell therapy dose can require billions of cells or trillions of viruses. As such, being able to provide a large quantity of cell or virus products in a short amount of time is critical for clinical success.

A significant portion of the cells used in bioprocessing are anchorage dependent, meaning the cells need a surface to adhere to for growth and functioning. Traditionally, the culturing of adherent cells is performed on two-dimensional (2D) cell-adherent surfaces incorporated in one of a number of vessel formats, such as T-flasks, petri dishes, cell factories, cell stack vessels, roller bottles, and HYPERStack® vessels. These approaches can have significant drawbacks, including the difficulty in achieving cellular density high enough to make it feasible for large scale production of therapies or cells.

Alternative methods have been suggested to increase volumetric density of cultured cells. These include microcarrier cultures performed in stir tanks. In this approach, cells that are attached to the surface of microcarriers are subject to constant shear stress, resulting in a significant impact on proliferation and culture performance. Another example of a high-density cell culture system is a hollow fiber bioreactor, in which cells may form large three-dimensional aggregates as they proliferate in the interspatial fiber space. However, the cells growth and performance are significantly inhibited by the lack nutrients. To mitigate this problem, these bioreactors are made small and are not suitable for large scale manufacturing

Another example of a high-density culture system for anchorage dependent cells is a packed-bed bioreactor system. In this this type of bioreactor, a cell substrate is randomly packed into a bioreactor vessel and used to provide a surface for the attachment of adherent cells. Media is perfused along the surface or through the packed bed to provide nutrients and oxygen needed for cell growth. For example, packed bed bioreactor systems that contain a packed bed to entrap the cells have been previously disclosed U.S. Pat. Nos. 4,833,083; 5,501,971; and 5,510,262. Packed bed matrices usually are made of porous particles as substrates or non-woven microfibers of polymer. Such bioreactors function as recirculation flow-through bioreactors. One of the significant issues with such bioreactors is the non-uniformity of cell distribution inside the packed bed. For example, the packed bed functions as depth filter with cells predominantly trapped at the inlet regions, resulting in a gradient of cell distribution during the inoculation step. In addition, due to random fiber packaging, flow resistance and cell trapping efficiency of cross sections of the packed bed are not uniform. For example, medium flows fast though the regions with low cell packing density and flows slowly through the regions where resistance is higher due to higher number of entrapped cells. This creates a channeling effect where nutrients and oxygen are delivered more efficiently to regions with lower volumetric cells densities and regions with higher cell densities are being maintained in suboptimal culture conditions.

Another significant drawback of packed bed systems disclosed in a prior art is the inability to efficiently harvest intact viable cells at the end of culture process. Harvesting of cells is important if the end product is cells, or if the bioreactor is being used as part of a “seed train,” where a cell population is grown in one vessel and then transferred to another vessel for further population growth. U.S. Pat. No. 9,273,278 discloses a bioreactor design to improve the efficiency of cell recovery from the packed bed during cells harvesting step. It is based on loosening the packed bed matrix and agitation or stirring of packed bed particles to allow porous matrices to collide and thus detach the cells. However, this approach is laborious and may cause significant cells damage, thus reducing overall cell viability.

For example, some packed bed bioreactors use small strips of cell substrate material consisting of randomly oriented fibers in a non-woven arrangement. These strips are packed into a vessel to create a packed bed. However, non-uniform packing of the substrate strips can create visible channels within the packed bed, leading to preferential and non-uniform media flow and nutrient distribution through the packed bed. Studies of such systems have noted a “systemic inhomogeneous distribution of cells, with their number increasing from top to bottom of fixed bed,” as well as a “nutrient gradient . . . leading to restricted cell growth and production,” all of which lead to the “unequal distribution of cells [that] may impair transfection efficiency.” (Rational plasmid design and bioprocess optimization to enhance recombinant adeno-associated virus (AAV) productivity in mammalian cells. Biotechnol. J. 2016, 11, 290-297). Studies have noted that agitation of the packed bed may improve dispersion, but would have other drawbacks (i.e., “necessary agitation for better dispersion during inoculation and transfection would induce increased shear stress, in turn leading to reduced cell viability.” Id.). Another study noted that the uneven distribution of cells makes monitoring of the cell population using biomass sensors difficult (“ . . . if the cells are unevenly distributed, the biomass signal from the cells on the top carriers may not show the general view of the entire bioreactor.” Process Development of Adenoviral Vector Production in Fixed Bed Bioreactor: From Bench to Commercial Scale. Human Gene Therapy, Vol. 26, No. 8, 2015).

In addition, because of the random arrangement of fibers in the substrate strips and the variation in packing of strips between one packed bed and another of a given packed bed reactor system, it can be difficult for users to predict cell culture performance, since the substrate varies between cultures. Furthermore, the packed substrate of many existing bioreactors makes efficiently harvesting cells very difficult or impossible, as it is believed that cells are entrapped by the packed bed.

There is a need for a platform that can produce high-quality cell and virus products in greater numbers in order to reach late-stage commercial manufacturing scale. In particular, there is a need for cell culture substrates and/or matrices, bioreactors, systems, and methods that enable culturing of cells in a high-density format, with uniform cell distribution, improved and uniform fluid flow characteristics, and easily attainable and increased harvesting yields.

### SUMMARY

According to embodiments of this disclosure, a cell culture matrix for culturing cells in a fixed bed reactor is provided. The cell culture matrix comprises a plurality of substrate layers in a stacked arrangement of parallel layers, each layer of the plurality of substrate layers comprising a substrate material with an ordered and regular array of openings passing through the layer. The openings are separated by the substrate material having a physical structure that is substantially regular and uniform and that is configured for growing cell thereon. The plurality of substrate layers comprises a first substrate material and a second substrate material that is different from the first substrate material in at least one physical dimension. The first substrate material and the second substrate material are separate layers of the plurality of substrate layers.

According to aspects of embodiments of this disclosure, the at least one physical dimension is at least one of a diameter of the openings, a thickness of the physical structure, a pattern of the physical structure, and a spacing of the physical structure on either side of an opening. The physical structure can comprise a plurality of fibers. In embodiments, the plurality of fibers comprises a first plurality of fibers running parallel to each other in a first direction, and a second plurality of fibers running parallel to each other in a second direction that is different from the first direction. In embodiments, the at least one physical dimension is a fiber spacing between two neighboring fibers of the plurality of fibers. A ratio of the fiber spacing of the first substrate material to the fiber spacing of the second substrate material is greater than 1.0, about 1.2 or greater, about 1.4 or greater, about 1.6 or greater, about 1.8 or greater, or from about 1.4 to about 1.8. A degree of relative rotation between the first substrate material and the second substrate material can be random. The first substrate material and the second substrate material are stacked as alternating layers of the plurality of substrate layers, according to aspects of some embodiments. In embodiments, variation in flow rate of fluid flowing through the cell culture matrix is uniform across a width of the cell culture matrix, the width being in a direction perpendicular to a direction of fluid flow.

According to embodiments of this disclosure, a bioreactor system for culturing cells is provided. The system comprises a cell culture vessel comprising at least one interior reservoir, an inlet fluidly connected to the reservoir, and an outlet fluidly connected to the reservoir; and the cell culture matrix described above, where the cell culture matrix is disposed in the reservoir.

According to aspects of embodiments, the bioreactor system is configured for perfusion flow through the cell culture matrix during cell culture. The bioreactor system can also be configured to harvest viable cells from the cell culture matrix within the reservoir.

According to embodiments of this disclosure, a cell culture matrix for culturing cells in a fixed bed reactor is provided, where the cell culture matrix comprises: a plurality of substrate layers in a stacked arrangement of parallel layers, each layer of the plurality of substrate layers comprising a plurality of interwoven fibers configured for culturing cells thereon, and an ordered and regular array of openings defined by the plurality of interwoven fibers and passing through the layer, wherein each layer of the plurality of substrate layers is rotated about a center of the layer relative to an immediately neighboring layer in the stack. According to aspects of embodiments, a degree of the rotation is about 20° or greater, about 20° to about 45°, about 30° to about 45°, or about 45°.

### BRIEF DESCRIPTION OF THE DRAWINGS

FIG. 1A shows a perspective view of a three-dimensional model of a cell culture substrate, according to one or more embodiments of this disclosure.

FIG. 1B is a two-dimensional plan view of the substrate of FIG. 1A.

FIG. 1C is a cross-section along line A-A of the substrate in FIG. 1B.

FIG. 2 shows a schematic view of a cell culture system with a fixed bed cell culture matrix, according to one or more embodiments.

FIG. 3A shows a plan view of a modeled multi-layer woven mesh cell culture substrate in an off-set or tightly packed arrangement, according to one or more embodiments of this disclosure.

FIG. 3B shows a side cross-section view of the multi-layer woven mesh cell culture substrate of FIG. 3A, according to one or more embodiments of this disclosure.

FIG. 4A shows a plan view of a modeled multi-layer woven mesh cell culture substrate in an aligned or loosely packed arrangement, according to one or more embodiments of this disclosure.

FIG. 4B shows a side cross-section view of the multi-layer woven mesh cell culture substrate of FIG. 4A, according to one or more embodiments of this disclosure.

FIG. 5A shows the modeled empty space in the dotted-line volume shown in FIGS. 3A and 3B.

FIG. 5B shows the modeled empty space in the dotted-line volume shown in FIGS. 4A and 4B.

FIG. 6A shows a schematic plan view of two layers of substrate superimposed or stacked in an aligned arrangement with a 0° relative rotation between the layers, according to embodiments of this disclosure.

FIG. 6B shows a schematic plan view of two layers of substrate superimposed or stacked in an off-set arrangement with a 0° relative rotation between the layers, according to embodiments of this disclosure.

FIG. 6C shows a schematic plan view of two layers of substrate superimposed or stacked with a 5° relative rotation between the layers, according to embodiments of this disclosure.

FIG. 6D shows a schematic plan view of two layers of substrate superimposed or stacked with a 10° relative rotation between the layers, according to embodiments of this disclosure.

FIG. 6E shows a schematic plan view of two layers of substrate superimposed or stacked with a 15° relative rotation between the layers, according to embodiments of this disclosure.

FIG. 6F shows a schematic plan view of two layers of substrate superimposed or stacked with a 20° relative rotation between the layers, according to embodiments of this disclosure.

FIG. 6G shows a schematic plan view of two layers of substrate superimposed or stacked with a 25° relative rotation between the layers, according to embodiments of this disclosure.

FIG. 6H shows a schematic plan view of two layers of substrate superimposed or stacked with a 30° relative rotation between the layers, according to embodiments of this disclosure.

FIG. 6I shows a schematic plan view of two layers of substrate superimposed or stacked with a 35° relative rotation between the layers, according to embodiments of this disclosure.

FIG. 6J shows a schematic plan view of two layers of substrate superimposed or stacked with a 40° relative rotation between the layers, according to embodiments of this disclosure.

FIG. 6K shows a schematic plan view of two layers of substrate superimposed or stacked with a 45° relative rotation between the layers, according to embodiments of this disclosure.

FIG. 7A is a schematic representation of fluid flow velocity before and after passing through a substrate layer, according to embodiments of this disclosure.

FIG. 7B is a graph of the flow velocity of fluid flowing through the substrate layer in FIG. 7A, according to embodiments of this disclosure.

FIG. 8 is a graph of the resistance of the porous substrate to the fluid flow in FIGS. 7A and 7B, according to embodiments of this disclosure.

FIG. 9 is a three-dimensional representation of a simplified model of the substrate material used in modeling flow through two layers of substrate, according to embodiments.

FIG. 10 is a graph of the simulated flow rate variation along a woven mesh surface using a sinusoidal model, according to embodiments of this disclosure.

FIG. 11A is a graph of the simulated flow rate variation along a two-layer woven mesh stack surface using a sinusoidal model for substrate layers and with an off-set arrangement and with a 0° relative rotation between the layers, according to embodiments of this disclosure.

FIG. 11B is a graph of the simulated flow rate variation along a two-layer woven mesh stack surface using a sinusoidal model for substrate layers with an aligned arrangement and with a 0° relative rotation between the layers, according to embodiments of this disclosure.

FIG. 12A shows two graphs of the simulated flow rate variation along the surface of a stack of two identical layers of woven mesh substrates at the boundary conditions of perfect alignment and off-set alignment, as shown in FIGS. 6A and 6B, respectively, with a 0° relative rotation between the layers, according to embodiments of this disclosure.

FIG. 12B shows a graph of the simulated flow rate variation along the surface of a stack of two identical layers of woven mesh substrates with a 5° relative rotation between the layers, according to embodiments of this disclosure.

FIG. 12C shows a graph of the simulated flow rate variation along the surface of a stack of two identical layers of woven mesh substrates with a 10° relative rotation between the layers, according to embodiments of this disclosure.

FIG. 12D shows a graph of the simulated flow rate variation along the surface of a stack of two identical layers of woven mesh substrates with a 20° relative rotation between the layers, according to embodiments of this disclosure.

FIG. 12E shows a graph of the simulated flow rate variation along the surface of a stack of two identical layers of woven mesh substrates with a 30° relative rotation between the layers, according to embodiments of this disclosure.

FIG. 12F shows a graph of the simulated flow rate variation along the surface of a stack of two identical layers of woven mesh substrates with a 45° relative rotation between the layers, according to embodiments of this disclosure.

FIG. 13A shows a graph of the simulated flow rate variation along the surface of a stack of two layers of woven mesh substrates having different filament spacing of a 1:1.2 ratio and with a 0° relative rotation between the layers, according to embodiments of this disclosure.

FIG. 13B shows a graph of the simulated flow rate variation along the surface of a stack of two layers of woven mesh substrates having different filament spacing of a 1:1.2 ratio and with a 5° relative rotation between the layers, according to embodiments of this disclosure.

FIG. 13C shows a graph of the simulated flow rate variation along the surface of a stack of two layers of woven mesh substrates having different filament spacing of a 1:1.2 ratio and with a 10° relative rotation between the layers, according to embodiments of this disclosure.

FIG. 13D shows a graph of the simulated flow rate variation along the surface of a stack of two layers of woven mesh substrates having different filament spacing of a 1:1.2 ratio and with a 20° relative rotation between the layers, according to embodiments of this disclosure.

FIG. 13E shows a graph of the simulated flow rate variation along the surface of a stack of two layers of woven mesh substrates having different filament spacing of a 1:1.2 ratio and with a 30° relative rotation between the layers, according to embodiments of this disclosure.

FIG. 13F shows a graph of the simulated flow rate variation along the surface of a stack of two layers of woven mesh substrates having different filament spacing of a 1:1.2 ratio and with a 45° relative rotation between the layers, according to embodiments of this disclosure.

FIG. 14A shows a graph of the simulated flow rate variation along the surface of a stack of two layers of woven mesh substrates having different filament spacing of a 1:1.4 ratio and with a 0° relative rotation between the layers, according to embodiments of this disclosure.

FIG. 14B shows a graph of the simulated flow rate variation along the surface of a stack of two layers of woven mesh substrates having different filament spacing of a 1:1.4 ratio and with a 5° relative rotation between the layers, according to embodiments of this disclosure.

FIG. 14C shows a graph of the simulated flow rate variation along the surface of a stack of two layers of woven mesh substrates having different filament spacing of a 1:1.4 ratio and with a 10° relative rotation between the layers, according to embodiments of this disclosure.

FIG. 14D shows a graph of the simulated flow rate variation along the surface of a stack of two layers of woven mesh substrates having different filament spacing of a 1:1.4 ratio and with a 20° relative rotation between the layers, according to embodiments of this disclosure.

FIG. 14E shows a graph of the simulated flow rate variation along the surface of a stack of two layers of woven mesh substrates having different filament spacing of a 1:1.4 ratio and with a 30° relative rotation between the layers, according to embodiments of this disclosure.

FIG. 14F shows a graph of the simulated flow rate variation along the surface of a stack of two layers of woven mesh substrates having different filament spacing of a 1:1.4 ratio and with a 45° relative rotation between the layers, according to embodiments of this disclosure.

FIG. 15A shows a graph of the simulated flow rate variation along the surface of a stack of two layers of woven mesh substrates having different filament spacing of a 1:1.6 ratio and with a 0° relative rotation between the layers, according to embodiments of this disclosure.

FIG. 15B shows a graph of the simulated flow rate variation along the surface of a stack of two layers of woven mesh substrates having different filament spacing of a 1:1.6 ratio and with a 5° relative rotation between the layers, according to embodiments of this disclosure.

FIG. 15C shows a graph of the simulated flow rate variation along the surface of a stack of two layers of woven mesh substrates having different filament spacing of a 1:1.6 ratio and with a 10° relative rotation between the layers, according to embodiments of this disclosure.

FIG. 15D shows a graph of the simulated flow rate variation along the surface of a stack of two layers of woven mesh substrates having different filament spacing of a 1:1.6 ratio and with a 20° relative rotation between the layers, according to embodiments of this disclosure.

FIG. 15E shows a graph of the simulated flow rate variation along the surface of a stack of two layers of woven mesh substrates having different filament spacing of a 1:1.6 ratio and with a 30° relative rotation between the layers, according to embodiments of this disclosure.

FIG. 15F shows a graph of the simulated flow rate variation along the surface of a stack of two layers of woven mesh substrates having different filament spacing of a 1:1.6 ratio and with a 45° relative rotation between the layers, according to embodiments of this disclosure.

FIG. 16A shows a graph of the simulated flow rate variation along the surface of a stack of two layers of woven mesh substrates having different filament spacing of a 1:1.8 ratio and with a 0° relative rotation between the layers, according to embodiments of this disclosure.

FIG. 16B shows a graph of the simulated flow rate variation along the surface of a stack of two layers of woven mesh substrates having different filament spacing of a 1:1.8 ratio and with a 5° relative rotation between the layers, according to embodiments of this disclosure.

FIG. 16C shows a graph of the simulated flow rate variation along the surface of a stack of two layers of woven mesh substrates having different filament spacing of a 1:1.8 ratio and with a 10° relative rotation between the layers, according to embodiments of this disclosure.

FIG. 16D shows a graph of the simulated flow rate variation along the surface of a stack of two layers of woven mesh substrates having different filament spacing of a 1:1.8 ratio and with a 20° relative rotation between the layers, according to embodiments of this disclosure.

FIG. 16E shows a graph of the simulated flow rate variation along the surface of a stack of two layers of woven mesh substrates having different filament spacing of a 1:1.8 ratio and with a 30° relative rotation between the layers, according to embodiments of this disclosure.

FIG. 16F shows a graph of the simulated flow rate variation along the surface of a stack of two layers of woven mesh substrates having different filament spacing of a 1:1.8 ratio and with a 45° relative rotation between the layers, according to embodiments of this disclosure.

### DETAILED DESCRIPTION

Various embodiments of the disclosure will be described in detail with reference to drawings, if any. Reference to various embodiments does not limit the scope of the invention, which is limited only by the scope of the claims attached hereto. Additionally, any examples set forth in this specification are not limiting and merely set forth some of the many possible embodiments of the claimed invention.

Embodiments of this disclosure include fixed bed cell culture substrates, as well as cell culture or bioreactor systems incorporating such a substrate. The substrates, and bioreactor systems incorporating the same, exhibit improved flow characteristics through the substrate. For example, more uniform flow is achieved through the substrate, and non-uniform flow resulting from channeling or turbulent flow is reduced or eliminated. Flow “dead zones” in the substrate or fixed bed of the bioreactor are greatly reduced or eliminated compared to alternative solutions. The result is a substrate or fixed bed that allows for uniform perfusion throughout the substrate or fixed bed, which promotes cell health during cell culture and an efficient cell culture process in terms of not only the culturing of cells, but also cell seeding, and harvesting of cells or cell by-products.

Embodiments of this disclosure also include fixed bed substrates and bioreactors that enable simplified and more efficient manufacturing and assembly. For example, embodiments include fixed bed cell culture substrates that must be assembled from one or more pieces of substrate, as well as bioreactors in which such fixed bed cell culture substrates are placed. Aspects of embodiments of this disclosure allow for such assembly of the fixed bed and/or placement of the fixed bed into reactor to be simplified by reducing the degree to which pieces of the cell culture substrate must be aligned with one another, which reduces the need for complicated procedures for handling and assembling the fixed bed, or the need for complicated mechanisms in the bioreactor to maintain particular orientations or alignments of the fixed bed. This reduction in complexity can translate to faster and cheaper manufacturing, shipping, and assembly, and more reliable bioreactors.

Aspects of embodiments also include fixed bed substrate matrices and bioreactors that provide more uniform fluid flow through the cell culture substrate fixed bed.

In conventional large-scale cell culture bioreactors, different types of fixed bed or packed bed bioreactors have been used. Usually these packed beds contain porous matrices to retain adherent or suspension cells, and to support growth and proliferation. Packed-bed matrices provide high surface area to volume ratios, so cell density can be higher than in the other systems. However, the packed bed often functions as a depth filter, where cells are physically trapped in the entangled fibers of the matrix. Thus, because of linear flow of the cell inoculum through the packed bed, cells are subject to heterogeneous distribution inside the packed bed, leading to variations in cell density through the depth or width of the packed bed. For example, cell density may be higher at the inlet region of a bioreactor and significantly lower nearer to the outlet of the bioreactor. This non-uniform distribution of the cells inside of the packed bed significantly hinders scalability and predictability of such bioreactors in bioprocess manufacturing, and can even lead to reduced efficiency in terms of growth of cells or viral vector production per unit surface area or volume of the packed bed.

Another problem encountered in packed bed bioreactors disclosed in prior art is the channeling effect. Due to random nature of packed nonwoven fibers, the local fiber density at any given cross section of the packed bed is not uniform. Liquid media flows quickly in the regions with low fiber density (high bed permeability) and much slower in the regions of high fiber density (lower bed permeability). The resulting non-uniform media perfusion across the packed bed creates the channeling effect, which manifests itself as significant nutrient and metabolite gradients that negatively impact overall cell culture and bioreactor performance. Cells located in the regions of low media perfusion will starve and very often die from the lack of nutrients or metabolite poisoning. The flow can be so non-uniform that there are effectively flow “dead zones” in the packed bed, where perfusion does not occur and the delivery of any nutrients to cells in those areas is limited to diffusion mechanics in the media.

Cell harvesting is yet another problem encountered when bioreactors packed with non-woven fibrous scaffolds are used. Due to packed-bed functions as depth filter, cells that are released at the end of cell culture process are entrapped inside the packed bed, and cell recovery is very low. This significantly limits utilization of such bioreactors in bioprocesses where live cells are the products or where live cells need to be harvested for further processing to capture cell by-products. Thus, the non-uniformity leads to areas with different exposure to flow and shear, effectively reducing the usable cell culture area, causing non-uniform culture, and interfering with transfection efficiency and cell release.

To address these and other problems of existing cell culture solutions, embodiments of the present disclosure provide cell growth substrates, fixed bed assemblies of such substrates, and/or bioreactor systems using such substrates that enable efficient and high-yield cell culturing for anchorage-dependent cells and production of cell products (e.g., proteins, antibodies, viral particles) with simplified manufacture and assembly of the fixed bed. Embodiments include a porous cell-culture matrix made from an ordered and regular array of porous substrate material that enables uniform cell seeding and media/nutrient perfusion, as well as efficient cell harvesting. Embodiments also enable scalable cell-culture solutions with substrates and bioreactors capable of seeding and growing cells and/or harvesting cell products from a process development scale to a full production size scale, without sacrificing the uniform performance of the embodiments. For example, in some embodiments, a bioreactor can be easily scaled from process development scale to product scale with comparable viral genome per unit surface area of substrate (VG/cm²) across the production scale. The harvestability and scalability of the embodiments herein enable their use in efficient seed trains for growing cell populations at multiple scales on the same cell substrate. In addition, the embodiments herein provide a cell culture matrix having a high surface area that, in combination with the other features described, enables a high yield cell culture solution. In some embodiments, for example, the cell culture substrate and/or bioreactors discussed herein can produce 10¹⁶ to 10¹⁸ viral genomes (VG) per batch.

In one embodiment, a matrix is provided with a structurally defined surface area for adherent cells to attach and proliferate that has good mechanical strength and forms a highly uniform multiplicity of interconnected fluidic networks when assembled in a packed bed or other bioreactor. In particular embodiments, a mechanically stable, non-degradable woven mesh can be used as the substrate to support adherent cell production. The cell culture matrix disclosed herein supports attachment and proliferation of anchorage dependent cells in a high volumetric density format. Uniform cell seeding of such a matrix is achievable, as well as efficient harvesting of cells or other products of the bioreactor. In addition, the embodiments of this disclosure support cell culturing to provide uniform cell distribution during the inoculation step and achieve a confluent monolayer or multilayer of adherent cells on the disclosed matrix, and can avoid formation of large and/or uncontrollable 3D cellular aggregates with limited nutrient diffusion and increased metabolite concentrations. Thus, the matrix eliminates diffusional limitations during operation of the bioreactor. In addition, the matrix enables easy and efficient cell harvest from the bioreactor. The structurally defined matrix of one or more embodiments enables complete cell recovery and consistent cell harvesting from the packed bed of the bioreactor.

In contrast to existing cell culture substrates used in cell culture bioreactors (e.g., non-woven substrates of randomly ordered fibers), embodiments of this disclosure include cell culture substrates having defined and ordered structures. The defined and order structure allows for consistent and predictable cell culture results. In addition, the substrates have open porous structures that prevent cell entrapment and enables uniform flow through the packed bed. This construction enables improved cell seeding, nutrient delivery, cell growth, and cell harvesting. According to one or more particular embodiments, the fixed bed matrix is formed with at least one substrate material having a thin, sheet-like construction having first and second sides separated by a relatively small thickness, such that the thickness of the sheet is small relative to the width and/or length of the first and second sides of the substrate. In addition, a plurality of holes or openings are formed through the thickness of the substrate. The substrate material between the openings is of a size and geometry that allows cells to adhere to the surface of the substrate material as if it were approximately a two-dimensional (2D) surface, while also allowing adequate fluid flow around the substrate material and through the openings. In some embodiments, the substrate is a polymer-based material, and can be formed as a molded polymer sheet; a polymer sheet with openings punched through the thickness; a number of filaments that are fused into a mesh-like layer; a 3D-printed substrate; or a plurality of filaments that are woven into a mesh layer. The physical structure of the fixed bed matrix has a high surface-to-volume ratio for culturing anchorage dependent cells. According to various embodiments, the fixed bed matrix can be arranged or packed in a bioreactor in certain ways discussed herein for uniform cell seeding and growth, uniform media perfusion, efficient cell harvest, and simplified manufacturing and packaging.

Embodiments of this disclosure can achieve viral vector platforms of a practical size that can produce viral genomes on the scale of greater than about 10¹⁴ viral genomes per batch, greater than about 10¹⁵ viral genomes per batch, greater than about 10¹⁶ viral genomes per batch, greater than about 10¹⁷ viral genomes per batch, or up to or greater than about g 10¹⁶ viral genomes per batch. In some embodiments, productions is about 10¹⁵ to about 10¹⁸ or more viral genomes per batch. For example, in some embodiments, the viral genome yield can be about 10¹⁵ to about 10¹⁶ viral genomes or batch, or about 10¹⁶ to about 10¹⁹ viral genomes per batch, or about 10¹⁶-10¹⁸ viral genomes per batch, or about 10¹⁷ to about 10¹⁹ viral genomes per batch, or about 10¹⁸ to about 10¹⁹ viral genomes per batch, or about 10¹⁸ or more viral genomes per batch.

In addition, the embodiments disclosed herein enable not only cell attachment and growth to a cell culture substrate, but also the viable harvest of cultured cells. The inability to harvest viable cells is a significant drawback in current platforms, and it leads to difficulty in building and sustaining a sufficient number of cells for production capacity. According to an aspect of embodiments of this disclosure, it is possible to harvest viable cells from the cell culture substrate, including between 80% to 100% viable, or about 85% to about 99% viable, or about 90% to about 99% viable. For example, of the cells that are harvested, at least 80% are viable, at least 85% are viable, at least 90% are viable, at least 91% are viable, at least 92% are viable, at least 93% are viable, at least 94% are viable, at least 95% are viable, at least 96% are viable, at least 97% are viable, at least 98% are viable, or at least 99% are viable. Cells may be released from the cell culture substrate using, for example, trypsin, TrypLE, or Accutase.

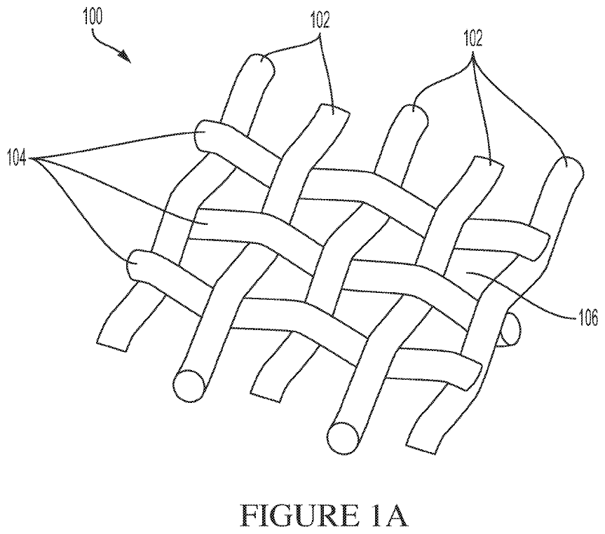

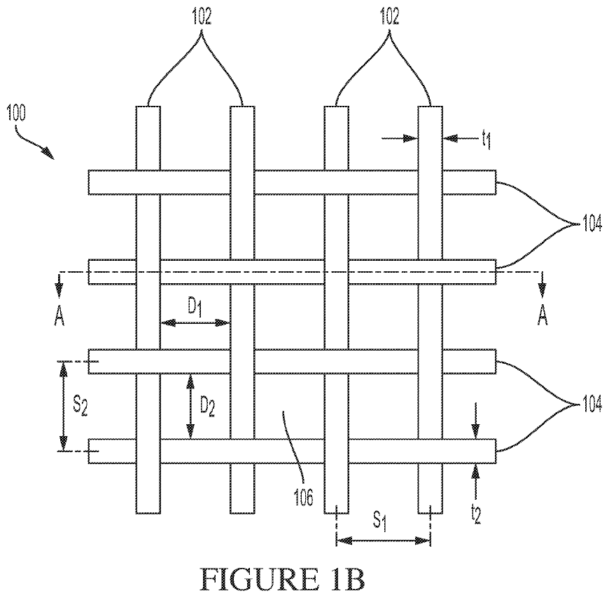

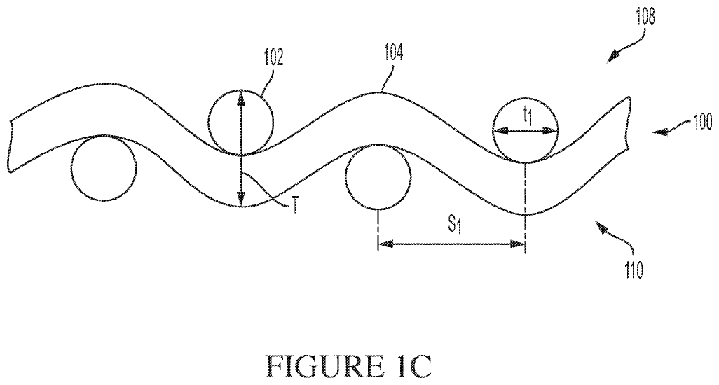

FIGS. 1A and 1B show a three-dimensional (3D) perspective view and a two-dimensional (2D) plan view, respectively, of a cell culture substrate 100, according to an example of one or more embodiments of this disclosure. The cell culture substrate 100 is a woven mesh layer made of a first plurality of fibers 102 running in a first direction and a second plurality of fibers 104 running in a second direction. The first direction and the second direction have a defined relationship to one another. For example, In FIGS. 1A and 1B, the first and second directions are perpendicular to one another. However, embodiments include other defined, non-random relative directions, such that the first and second directions can be separated by some angle less than 90°, including, for example, 30°, 45°, 60°, or 75°, or any other defined angle. The first plurality of fibers 102 are spaced from one another by a first fiber spacing S₁, and the second plurality of fibers 104 are spaced from one another by a second fiber spacing S₂. The first and second fiber spacings 102 and 104 are defined by the perpendicular distance from the center line of one fiber to the center line of an adjacent or nearest fiber in the first and second plurality of fibers 102 and 104, respectively. According to various embodiments, the first and second fiber spacings S₁ and S₂ may be equal or unequal, as discussed below.

The woven fibers of the substrate 100 form a plurality of openings 106, which can be defined by one or more widths or diameters (e.g., D₁, D₂). The size and shape of the openings can vary based on the type of weave (e.g., number, shape and size of filaments; angle between intersecting filaments, etc.). A woven mesh may be characterized as, on a macro-scale, a two-dimensional sheet or layer. However, a close inspection of a woven mesh reveals a three-dimensional structure due to the rising and falling of intersecting fibers of the mesh. Thus, as shown in FIG. 1C, a thickness T of the woven mesh 100 may be thicker than the thickness of a single fiber (e.g., t₁). As used herein, the thickness T is the maximum thickness between a first side 108 and a second side 110 of the woven mesh. Without wishing to be bound by theory, it is believed that the three-dimensional structure of the substrate 100 is advantageous as it provides a large surface area for culturing adherent cells, and the structural rigidity of the mesh can provide a consistent and predictable cell culture matrix structure that enables uniform fluid flow.

In FIG. 1B, the openings 106 have a diameter D₁, defined as a distance between opposite fibers of the first plurality of fibers 102, and a diameter D₂, defined as a distance between opposite fibers of the second plurality of fibers 104. D₁ and D₂ can be equal or unequal, depending on the weave geometry. Where D₁ and D₂ are unequal, the larger can be referred to as the major diameter, and the smaller as the minor diameter. In some embodiments, the diameter of an opening may refer to the widest part of the opening. Unless otherwise specified, the opening diameter, as used herein, will refer to a distance between parallel fibers on opposite sides of an opening.

A given fiber of the first plurality of fibers 102 has a thickness t₁, and a given fiber of the second plurality of fibers 104 has a thickness t₂. In the case of fibers of round cross-section, as shown in FIG. 1A, or other three-dimensional cross-sections, the thicknesses t₁ and t₂ are the maximum diameters or thicknesses of the fiber cross-section. According to some embodiments, the first plurality of fibers 102 all have the same thickness t₁, and the second plurality of fiber 104 all have the same thickness t₂. In addition, t₁ and t₂ may be equal. However, in one or more embodiments, t₁ and t₂ are not equal such as when the first plurality of fibers 102 are different from the second plurality of fibers 104. In addition, each of the first plurality of fibers 102 and the second plurality of fibers 104 may contain fibers of two or more different thicknesses (e.g., t₁ₐ, t_{1b}, etc., and t₂ₐ, t_{2b}, etc.). According to embodiments, the thicknesses t₁ and t₂ are large relative to the size of the cells cultured thereon, so that the fibers provide an approximation of a flat surface from the perspective of the cell, which can enable better cell attachment and growth as compared to some other solutions in which the fiber size is small (e.g., on the scale of the cell diameter). Due to three-dimensional nature of woven mesh, as shown in FIGS. 1A-1C, the 2D surface area of the fibers available for cell attachment and proliferation exceeds the surface area for attachment on an equivalent planar 2D surface.

In one or more embodiments, a fiber may have a diameter in a range of about 10 μm to about 1000 μm; about 10 μm to about 750 μm; about 15 μm to about 600 μm; about 150 μm to about 500 μm; about 20 μm to about 400 μm; about 30 μm to about 325 μm; from about 15 μm to about 200 μm; or about 150 μm to about 300 μm. On a microscale level, due to the scale of the fiber compared to the cells (e.g., the fiber diameters being larger than the cells), the surface of monofilament fiber is presented as an approximation of a 2D surface for adherent cells to attach and proliferate. Fibers can be woven into a mesh with openings ranging from about 100 μm×100 μm to about 1000 μm×1000 μm. In some embodiments, the opening may have a diameter of about 20 μm to about 1000 μm; about 100 μm to about 750 μm; about 125 μm to about 600 μm; about 150 μm to about 500 μm; about 200 μm to about 400 μm; or about 200 μm to about 300 μm; from about 40 μm to about 900 μm, from about 50 μm to about 300 μm, or from about 225 μm to about 800 μm. These ranges of the filament diameters and opening diameters are examples of some embodiments, but are not intended to limit the possible feature sizes of the mesh according to all embodiments. The combination of fiber diameter and opening diameter is chosen to provide efficient and uniform fluid flow through the substrate when, for example, the fixed bed cell culture matrix comprises a number of adjacent mesh layers (e.g., a stack of individual layers or a rolled mesh layer).

Factors such as the fiber diameter, opening diameter and/or fiber spacing, and weave type/pattern will determine the surface area available for cell attachment and growth. In addition, when the cell culture matrix includes a stack, roll, or other arrangement of overlapping substrate, the packing density of the cell culture matrix will impact the surface area of the packed bed matrix. Packing density can vary with the packing thickness of the substrate material (e.g., the space needed for a layer of the substrate). For example, if a stack of cell culture matrix has a certain height, each layer of the stack can be said to have a packing thickness determined by dividing the total height of the stack by the number of layers in the stack. The packing thickness will vary based on fiber diameter and weave, but can also vary based the alignment of adjacent layers in the stack. Due to the three-dimensional nature of a woven layer, there is a certain amount of interlocking or overlapping that can occur between adjacent layers based on their alignment with one another. In a first alignment, the adjacent layers can be tightly nestled together, but in a second alignment, the adjacent layers can have zero overlap, such as when the lower-most point of the upper layer is in direct contact with the upper-most point of the lower layer. In practice, the amount of nesting can be impacted by both the translational and rotational alignment of the fiber patterns in adjacent layers. It may be desirable for certain applications to provide a cell culture matrix with a lower density packing of layers (e.g., when higher permeability is a priority) or a higher density of packing (e.g., when maximizing substrate surface area is a priority). According to one or more aspects of embodiments herein, the packing thickness can be from about 50 μm to about 1000 μm; about 100 μm to about 750 μm; about 125 μm to about 600 μm; about 150μm to about 500 μm; about 200 μm to about 400 μm; about 200 μm to about 300 μm. There ranges are provided by way of example only, and embodiments can include packing thicknesses outside of these ranges based on the chosen substrate, packing of the fixed bed, bioreactor design, and application.

The above structural factors can determine the available surface area of a cell culture matrix, whether of a single layer of cell culture substrate or of a cell culture matrix having multiple layers of substrate. The “effective surface area,” as used herein, is the total surface area of fibers in a portion of substrate material that is available for cell attachment and growth. Unless stated otherwise, references to “surface area” refer to this effective surface area. According to embodiments, a single woven mesh substrate layer with a diameter of, for example, 6 cm may have an effective surface area of about 50 cm² to about 90 cm²; about 53 cm² to about 81 cm²; about 68 cm²; about 75 cm²; or about 81 cm², depending on the fiber size, arrangement of fibers/openings within the substrate, and obviously the size of the substrate layer itself. These ranges of effective surface area are provided for example only, and some embodiments may have different effective surface areas. The cell culture matrix can also be characterized in terms of porosity, as discussed in the Examples herein.

The substrate mesh can be fabricated from monofilament or multifilament fibers of polymeric materials compatible in cell culture applications, including, for example, polystyrene, polyethylene terephthalate, polycarbonate, polyvinylpyrrolidone, polybutadiene, polyvinylchloride, polyethylene oxide, polypyrroles, and polypropylene oxide. Mesh substrates may have a different patterns or weaves, including, for example knitted, warp-knitted, or woven (e.g., plain weave, twilled weave, dutch weave, five needle weave). Aspects of embodiments also include filaments made of any other suitable material for forming the porous structure and that are then coated with materials compatible with cell culture applications.

The surface chemistry of the mesh filaments may need to be modified to provide desired cell adhesion properties. Such modifications can be made through the chemical treatment of the polymer material of the mesh or by grafting cell adhesion molecules to the filament surface. Alternatively, meshes can be coated with thin layer of biocompatible hydrogels that demonstrate cell adherence properties, including, for example, collagen or Matrigel®. Alternatively, surfaces of filament fibers of the mesh can be rendered with cell adhesive properties through the treatment processes with various types of plasmas, process gases, and/or chemicals known in the industry. In one or more embodiments, however, the mesh is capable of providing an efficient cell growth surface without surface treatment.

The three-dimensional quality of the substrates according to embodiments of this disclosure provides increased surface area for cell attachment and proliferation compared to a planar 2D surface of comparable size. This increased surface area aids in the scalable performance achieved by embodiments of this disclosure. For process development and process validation studies, small-scale bioreactors are often required to save on reagent cost and increase experimental throughput. Embodiments of this disclosure are applicable to such small-scale studies, but can be scaled-up to industrial or production scale, as well. Because of the plug-type perfusion flow in a packed bed, the same flow rate expressed in ml/min/cm² of cross-sectioned packed bed surface area can be used in smaller-scale and larger-scale versions of the bioreactor. A larger surface area allows for higher seeding density and higher cell growth density. According to one or more embodiments, the cell culture substrate described herein has demonstrated cell seeding densities of up to 22,000 cells/cm² or more. For reference, the Corning HyperFlask® has a seeding density on the order of 20,000 cells/cm² on a two-dimensional surface. As used herein, “plug-type perfusion flow” or “plug flow” refers to laminar flow through the bioreactor having a fixed bed according to embodiments herein, where the flow through any cross-section of the fixed bed perpendicular to the flow direction proceeds at the same rate across the cross section.

Another advantage of the higher surface areas and high cell seeding or growing densities is that the cost of the embodiments disclosed herein can be the same or less than competing solution. Specifically, the cost per cellular product (e.g., per cell or per viral genome) can be equal to or less than other packed bed bioreactors.

Thus, there are multiple factors of the fixed bed substrate that can impact the cell culture process, including the packing density, surface chemistry, and effective surface area, as well as the nature of fluid flow through and within the packed bed. By using a structurally defined culture matrix of sufficient rigidity, high-flow-resistance uniformity across the matrix or packed bed is achieved. According to various embodiments, the matrix can be deployed in flexible and scalable multilayer substrate arrangements. This flexibility eliminates diffusional limitations and provides uniform delivery of nutrients and oxygen to cells attached to the matrix. In addition, the open matrix lacks any cell entrapment regions in the packed bed configuration, allowing for complete cell harvest with high viability at the end of culturing. The matrix also delivers packaging uniformity for the packed bed, and enables direct scalability from process development units to large-scale industrial bioprocessing unit. The ability to directly harvest cells from the packed bed eliminates the need of resuspending a matrix in a stirred or mechanically shaken vessel, which would add complexity and can inflict harmful shear stresses on the cells. Further, the high packing density of the cell culture matrix yields high bioprocess productivity in volumes manageable at the industrial scale.

The geometry of the mesh substrate layers is designed to allow efficient and uniform flow through one or multiple substrate layers. In addition, the structure of the matrix can accommodate fluid flow through the matrix in multiple orientations. For example, the direction of bulk fluid flow can be perpendicular to the major side surfaces of the first and second substrate layers. However, the matrix can also be oriented with respect to the flow such that the sides of the substrate layers are parallel to the bulk flow direction. In addition to fluid flow being perpendicular or parallel to the first and second sides of the mesh layers, the matrix can be arranged with multiple pieces of substrate at intermediate angles, or even in random arrangements with respect to fluid flow. This flexibility in orientation is enabled by the essentially isotropic flow behavior of the woven substrate. In contrast, substrates for adherent cells in existing bioreactors do not exhibit this behavior and instead their packed beds tend to create preferential flow channels and have substrate materials with anisotropic permeability. The flexibility of the matrix of the current disclosure allows for its use in various applications and bioreactor or container designs while enabling better and more uniform permeability throughout the bioreactor vessel.

The cell culture substrate can be used within a bioreactor vessel, according to one or more embodiments. For example, the substrate can be used in a packed bed bioreactor configuration, or in other configurations within a three-dimensional culture chamber. However, embodiments are not limited to a three-dimensional culture space, and it is contemplated that the substrate can be used in what may be considered a two-dimensional culture surface configuration, where the one or more layers of the substrate lay flat, such as within a flat-bottomed culture dish, to provide a culture substrate for cells. Due to contamination concerns, the vessel can be a single-use vessel that can be disposed of after use.

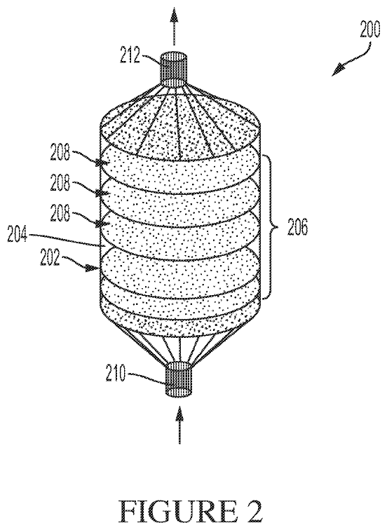

A cell culture system is provided, according to embodiments, in which the cell culture matrix is used within a culture chamber of a bioreactor vessel. FIG. 2 shows an example of a cell culture system 200 that includes a bioreactor vessel 202 having a cell culture chamber 204 in the interior of the bioreactor vessel 202. Within the cell culture chamber 204 is a cell culture matrix 206 that is made from a stack of substrate layers 208. The substrate layers 208 are stacked with the first or second side of a substrate layer facing a first or second side of an adjacent substrate layer. The bioreactor vessel 200 has an inlet 210 at one end for the input of media, cells, and/or nutrients into the culture chamber 204, and an outlet 212 at the opposite end for removing media, cells, or cell products from the culture chamber 204. By allowing stacking of substrate layers in this way, the system can be easily scaled up without negative impacts on cell attachment and proliferation, due to the defined structure and efficient fluid flow through the stacked substrates. While the vessel 200 may generally be described as having an inlet 210 and an outlet 212, some embodiments may use one or both of the inlet 210 and outlet 212 for flowing media, cells, or other contents both into and out of the culture chamber 204. For example, inlet 210 may be used for flowing media or cells into the culture chamber 204 during cell seeding, perfusion, or culturing phases, but may also be used for removing one or more of media, cells, or cell products through the inlet 210 in a harvesting phase. Thus, the terms “inlet” and “outlet” are not intended to restrict the function of those openings.

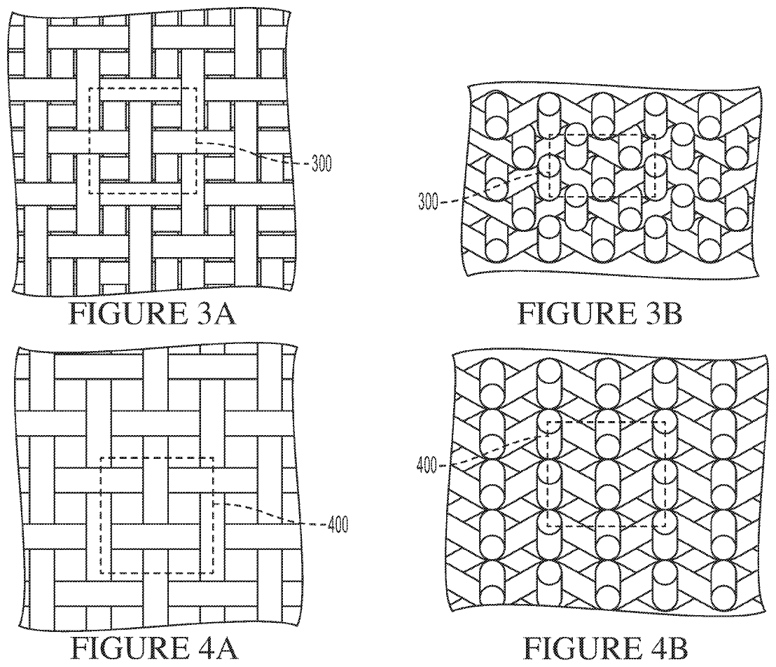

In FIG. 2, the bulk flow direction is in a direction from the inlet 210 to the outlet 212, and, in this example, the first and second major sides of the substrate layers 208 are perpendicular to the bulk flow direction. According to some aspects of embodiments, the substrates can be disposed in other configurations than that shown in FIG. 2, which is shown as an example only. In bioreactor system 200, the substrates 208 are sized and shaped to fill the interior space defined by the culture chamber 204 so that the culture space in the vessel is filled for cell growth surfaces to maximize efficiency in terms of cells per unit volume. Although FIG. 3 shows a single inlet 210 and a single outlet 212, it is contemplated that the system 200 may be fed by multiple inlets and have multiple outlets. According to embodiments herein, distribution plates can be used to help distribute the media, cells, or nutrients across a cross-section of the packed bed and thus improve uniformity of fluid flow through the packed bed.

The cell culture matrix can be arranged in multiple configurations within the culture chamber depending on the desired system. For example, in one or more embodiments, the system includes one or more layers of the substrate with a width extending across the width of a defined cell culture space in the culture chamber. Multiple layers of the substrate may be stacked in this way to a predetermined height. As discussed above, the substrate layers may be arranged such that the first and second sides of one or more layers are perpendicular to a bulk flow direction of culture media through the defined culture space within the culture chamber, or the first and second sides of one or more layers may be parallel to the bulk flow direction. In one or more embodiments, the cell culture matrix includes one or more substrate layers at a first orientation with respect to the bulk flow, and one or more other layers at a second orientation that is different from the first orientation. For example, various layers may have first and second sides that are parallel or perpendicular to the bulk flow direction, or at some angle in between.

In one or more embodiments, the cell culture system includes a plurality of discrete pieces of the cell culture substrate in a packed bed configuration, where the length and or width of the pieces of substrate are small relative to the culture chamber. As used herein, the pieces of substrate are considered to have a length and/or width that is small relative to the culture chamber when the length and/or width of the piece of substrate is about 50% or less of the length and/or width of the culture space. Thus, the cell culture system may include a plurality of pieces of substrate packed into the culture space in a desired arrangement. The arrangement of substrate pieces may be random or semi-random, or may have a predetermined order or alignment, such as the pieces being oriented in a substantially similar orientation (e.g., horizontal, vertical, or at an angle between 0° and 90° relative to the bulk flow direction).

The “defined culture space,” as used herein, refers to a space within the culture chamber occupied by the cell culture matrix and in which cell seeding and/or culturing is to occur. The defined culture space can fill approximately the entirety of the culture chamber, or may occupy a portion of the space within the culture chamber. As used herein, the “bulk flow direction” is defined as a direction of bulk mass flow of fluid or culture media through or over the cell culture matrix during the culturing of cells, and/or during the inflow or outflow of culture media to the culture chamber.

In one or more embodiments, the cell culture matrix is secured within the culture chamber by a fixing mechanism. The fixing mechanism may secure a portion of the cell culture matrix to a wall of the culture chamber that surrounds the matrix, or to a chamber wall at one end of the culture chamber. In some embodiments, the fixing mechanism adheres a portion of the cell culture matrix to a member running through the culture chamber, such as member running parallel to the longitudinal axis of the culture chamber, or to a member running perpendicular to the longitudinal axis. However, in one or more other embodiments, the cell culture matrix may be contained within the culture chamber without being fixedly attached to the wall of the chamber or bioreactor vessel. For example, the matrix may be contained by the boundaries of the culture chamber or other structural members within the chamber such that the matrix is held within a predetermined area of the bioreactor vessel without the matrix being fixedly secured to those boundaries or structural members.

When multiple layers of substrate are stacked face-to-face as shown in FIG. 2, the packing density of the layers can vary based on the alignment and degree of nesting or overlap among the fibers of adjacent layers. For example, assuming no relative rotation between adjacent layers, in which case the first plurality of fibers of one layer are parallel to the first plurality of fibers of another layer, the packing density boundary conditions are defined by the amount of nesting or overlap between adjacent layers. If the fibers of adjacent layers are not perfectly aligned, there will be some degree of nesting of fibers from one layer among fibers of the adjacent layer due to the three-dimensional nature of substrates, according to one or more embodiments. FIGS. 3A and 3B show a plan view and cross-section view, respectively, of this scenario. Specifically, the fibers of a first layer running in a first direction are shifted halfway between fibers of an adjacent second layer that run in the first direction, such that the fibers of the first layer rest in the openings of the second layer (see FIG. 3A). This represents the highest degree of overlap that can occur between adjacent layers, and thus the highest degree of compaction (without some additional external force to squeeze the layers together). On the other end of the spectrum, FIGS. 4A and 4B show similar views of a stack of substrate layers in which fibers running in a first direction of one substrate layer are all perfectly aligned with fibers running in the first direction in other substrate layers. This represents the lowest degree of overlap (i.e., zero overlap), and thus the lowest degree of compaction while the layers are still in contact with each other.

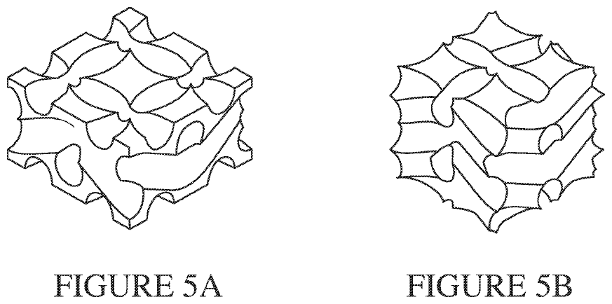

For each modeled configuration in FIGS. 3A-4B, a sample cell 300, 400 was defined that encloses the same volume of mesh material to analyze the porosity per unit volume of the sample cell 300, 400. The modeled volume of open space within each cell is shown in FIGS. 5A (for the tight-packed stack) and 5B (for the loose-packed stack). The porosity in terms of percentage of open space was about 40.8% for the loose-packed cell, and 61.4% for the tight-packed cell. Because the modeled stacks in FIGS. 3A-4B represent the tightest-and loosest-packed configurations for the given mesh material, the porosities of 40.8% and 61.4% are the upper and lower bounds of porosity for this particular mesh material. Depending on the alignment and real-world packing density when using this mesh material, the porosity may fall in between these extremes. However, embodiments of this disclosure are not limited to this porosity range, as variations in the mesh dimensions and arrangement of the substrate within the cell culture vessel can lead to a different range of porosities.

In addition to the modeled porosity range, porosity was measured using real packed beds of PET woven mesh substrate. The measurements were made using one hundred disks, each with a diameter of 22.4 mm, stacked with random alignment. The total weight of the 100-disk stack was 5.65±0.2 g. Volume of the PET material of the stack was calculated, assuming a PET density of 1.38 g/cm³, using the following formula:

$${\begin{matrix}{V}_{PET} = ( total weight of stack ) / ( density of PET ) & Equation l\end{matrix}}_{}$$

Thus, the PET volume V_{PET} of 5.65 g of PET (for 100 disks of 22.4 mm diameter) was calculated to be 4.1 ml. The total volume Vₜₒₜₐₗ of the stack, including the PET volume V_{PET} and the volume of the open space within the stack, was then calculated using the following formula:

$${V}_{total} = π \times ( 0.5 \times disk diameter ) \times ( stacked bed height ) \qquad Equation 2$$

The 100-disk stack had a stack height of 25±1 mm. Thus, with a disk diameter of 22.4 mm, Vₜₒₜₐₗ was found to be 9.85 ml. Accordingly, porosity of the stacked bed can be calculated using the following:

$$Porosity = ( {V}_{total} - {V}_{PET} ) / {V}_{t otal} \qquad Equation 3$$

Using Equation 3 and the above values, the porosity was calculated to be 58.4%, which is within the range predicted by the model.

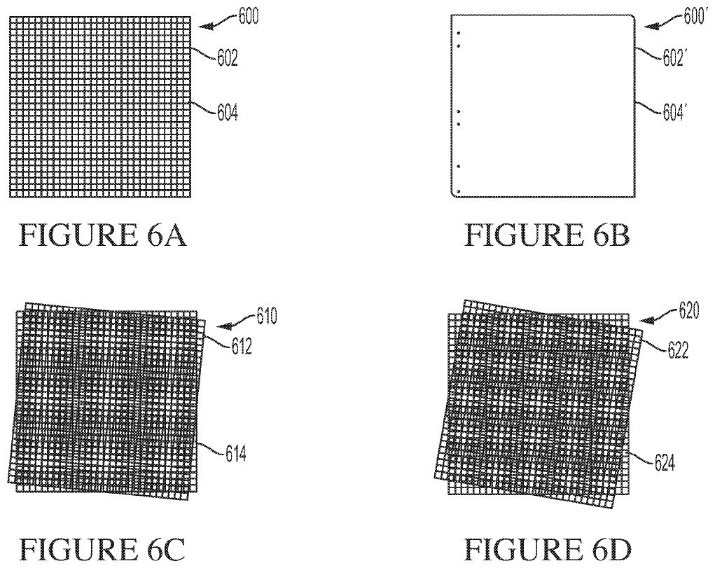

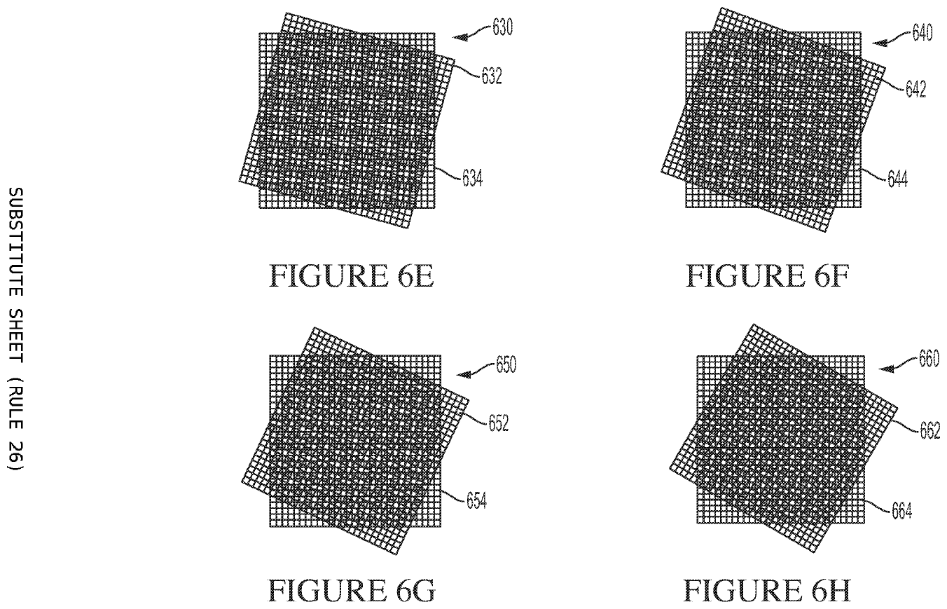

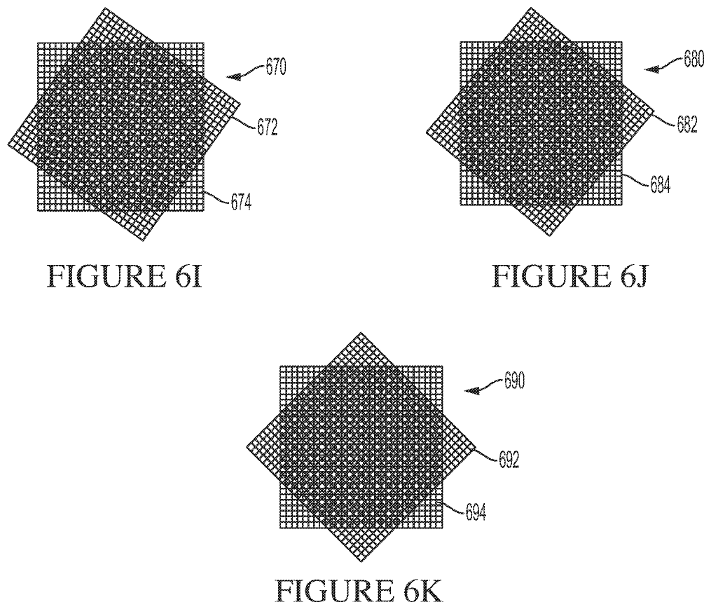

As discussed above, in the loose-packed configuration, fibers in layers of the fixed bed are aligned. It follows that the openings in the layers will also be aligned if the substrate materials in the layers are the same. Thus, if viewing a stack of substrates in this loose-packed configuration in plan view, it will be possible to see through the aligned openings in the stack. FIG. 6A shows such a stack of loose-packed substrate layers 600, having at least two layers 602, 604. On the other hand, in the tight-packed configuration, it is not possible to see through the stack when viewed from above due to the fibers of one layer overlapping with the openings of an adjacent layer. FIG. 6B represents this configuration with stack 600′ having at least two layers 602′, 604′ that are perfectly nested. For a fixed bed made of woven mesh, the alignment of fibers between the neighboring layers has been shown to have a significant effect on the flow uniformity through the bed. For layers with a rotational alignment of 0° offset, the difference in permeability of stacked substate layers completely interlocked as shown in FIG. 4A can be as large as ten times (10×) as compared to when the layers completely overlap as shown FIG. 3A.

However, in addition to translational shifts between layers in a fixed bed, it is also possible for rotational misalignment. It should be noted that both translational and rotational alignment can be factures of the fabrication of the substrate material itself (e.g., when individual layers are cut from a larger sheet) or from shifting between layers in the fixed bed (e.g., shifting that can occur during handling or assembly of a bioreactor, or once the fixed bed bioreactor is fully assembled). While strict manufacturing and assembling tolerances can accommodate for and reduce such shifts, such precision adds complexity and cost to the process.

To appreciate the impact that rotational alignment of substrate layers can have on the fixed bed properties, FIGS. 6C-6K show substrate stacks with various degrees of rotation between two layers, and the possible effects that such rotation has on the fluid flow performance due to the degree to the stacks have uniform openings available for fluid flow. In FIG. 6C, the layer 612 is rotated 5° relative to layer 614 in the stack 610; in FIG. 6D, the layer 622 is rotated 10° relative to layer 624 in the stack 620; FIG. 6E, the layer 632 is rotated 15° relative to layer 634 in the stack 630; FIG. 6F, the layer 642 is rotated 20° relative to layer 644 in the stack 640; FIG. 6G, the layer 652 is rotated 25° relative to layer 654 in the stack 650; FIG. 6H, the layer 662 is rotated 30° relative to layer 664 in the stack 660; FIG. 6I, the layer 672 is rotated 35° relative to layer 674 in the stack 670; FIG. 6J, the layer 682 is rotated 40° relative to layer 684 in the stack 680; and FIG. 6K, the layer 692 is rotated 45° relative to layer 694 in the stack 690.

If there is an angle of rotation between two layers, particularly for the lower angles such as 5° to 10° in FIGS. 6C and 6D, some clear patterns can be observed in the openings of the stack. There are definitive dark and light regions, which represents significant differences in permeability between those regions. For example, the lighter regions are due to consecutive openings being overlaid with the openings in the neighboring layer. Thus, one would expect less resistances to flow in those regions. In other regions where consecutive openings are blocked by fibers from the neighbor layer, the stack appears dark and one would expect those dark regions to have more resistance to flow. This suggests that those different regions will have different flow rates. Different flow rates can potentially cause different cell seeding and nutrients supply, and therefore non-uniform cell growth. It may also affect access of transfection reagents during transfection and shear force during harvesting. Directly measuring or quantifying this non-uniform distribution of flow is challenging. However, non-uniform uniform distribution of cells after staining the mesh after cell culture has been observed in these situations. The staining images showed similar patterns suggesting that non-uniform flow due to different alignment of layers does exist and can negatively impact cell culture uniformity. To decrease such uniformity, alignments with relatively uniform dark-light regions are preferred. From FIGS. 6A-6K, rotational angles of from about 30° to about 45° are preferable (see FIGS. 6H-6K), with 45° (FIG. 6K) being a particularly preferred embodiment.

According to embodiments, a cell culture matrix is provided where each layer of the plurality of substrate layers is rotated about the center of the layer relative to an immediately neighboring layer in the stack such that an orientation of fibers in one layer of the plurality of substrate layers is different from an orientation of fibers in an immediately adjacent layer of the plurality of substrate layers. The difference in orientation can be measured by an angle of rotation of the fibers in one layer relative to another layer. A difference in the orientation of fibers in the one layer and the immediately adjacent layer can be, in some embodiments, about 5° or more, about 10° or more, about 15° or more, about 20° or more, about 25° or more, about 30° or more, or about 40° or more, and less than about 90°, about 85° or less, about 80° or less, about 75° or less, about 65° or less, about 60° or less, or about 50° or less. In some particular embodiments, the difference in the orientation is about 45°. In embodiments, the difference in the orientation is from about 40° to about 50°.

However, the precise degree of rotational alignment described above can be difficult. It requires steps be taken to align the mesh during reactor assembly and mechanical features to hold the alignment during handling, shipping and application. These extra steps and mechanical features will add extra costs and risks. Therefore, embodiments of this disclosure include fixed beds for cell culture that achieve uniform flow without requiring these costly measures to ensure precise rotational alignment.

According to embodiments of this disclosure, a fixed bed is provided having at least two different types of substrate materials stacked together in the same cell culture bed. In embodiments, the at least two different substrates are alternately stacked (e.g., Substrate A, Substrate B, Substrate A, Substate B, etc.). The at least two different types of substrate material can be different in one or more physical dimensions. For example, they may be different in fiber diameter, opening diameter, fiber spacing, or fiber direction. Embodiments using different types of substate material provide several advantages, including: no specific alignment is required during reactor assembly; no mechanical features are required in the vessel to hold the mesh in place; minimized variation in packed bed porosity or density; and improved flow uniformity in a packed bed reactor. The term “hybrid substrate,” “hybrid mesh,” or “hybrid fixed bed” are sometimes used herein to refer to a substrate matrix or fixed bed that contains at least two different types of substrate material, as discussed above.

In one or more preferred embodiments, the two different types of substrate have a different fiber spacing. The relationship of the fiber spacings of two different can be expressed as a ratio of the fiber spacing of the first mesh to the fiber spacing of the second mesh. Embodiments include at least two types of substrate of mesh with a fiber spacing ratio of at least about 1.1 and at most about 2.0, 2.5, 3.5, 4.0, 4.5, and 5.0; of a fiber spacing ratio of about 1.2 to about 4.0; or about 1.2 to about 2.0. According to embodiments with a fiber spacing ratio, it is not necessary to control or hold the layers to maintain a specific alignment. This can be demonstrated by modeling the flow patterns through stacks of substrate layers with different fiber spacing ratios, for example, as discussed below.

### EXAMPLE

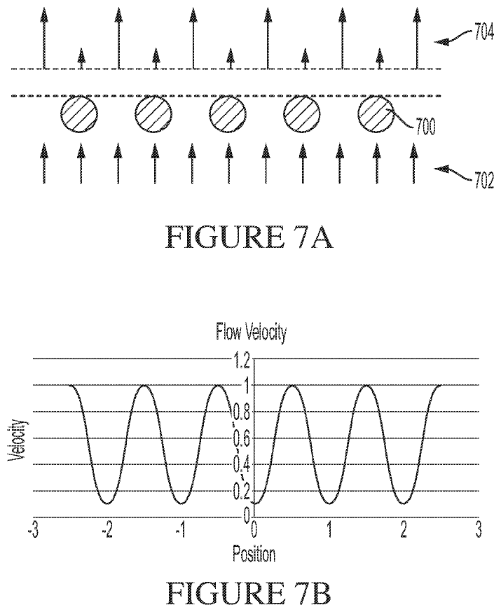

To demonstrate the impact of mesh alignment on flow uniformity and the benefit of using hybrid meshes, a simplified sinusoidal model can simulate the periodical change of flow resulting from passing a layer of mesh. The mesh is a woven pattern of two groups of parallel fibers (a first group of fibers running in parallel in a first direction, and a second group of fibers running in parallel in a second direction). As a basic model, a serial parallel cylinder is used to simulate the first group of fibers running in a first direction. FIG. 7A shows a cross-section of a series of such fibers 700. In this example, the cylinders have a diameter of 160 μm (the fiber diameter) and an opening diameter of 250 μm between each cylinder. Water 702 flowing at an average rate of 30 ml/min toward the cylinders, which is within a typical range commonly used in cell culture, passes through the series of cylinders 700 and will have a different velocity profile 704 on the other side of the cylinders due to the obstruction and resistance from the cylinders. Using a computational fluid dynamics (CFD) model, the water velocity follows a sinusoidal function at 160 μm away on the other side of the array, as shown in FIG. 7B.

The resistance of a porous material, such as the series of cylinders 700, to fluid flow can be calculated from the flow rate using Equation 4, which is proportional to the reciprocal of flow rate, where R is the flow resistance, P is the pressure, and U is the flow rate. This reciprocal of the sinusoidal equation is used to represent the relative changes of flow resistance at different locations along the cylinder array.

$$R = \frac{P}{U} \qquad Equation 4$$

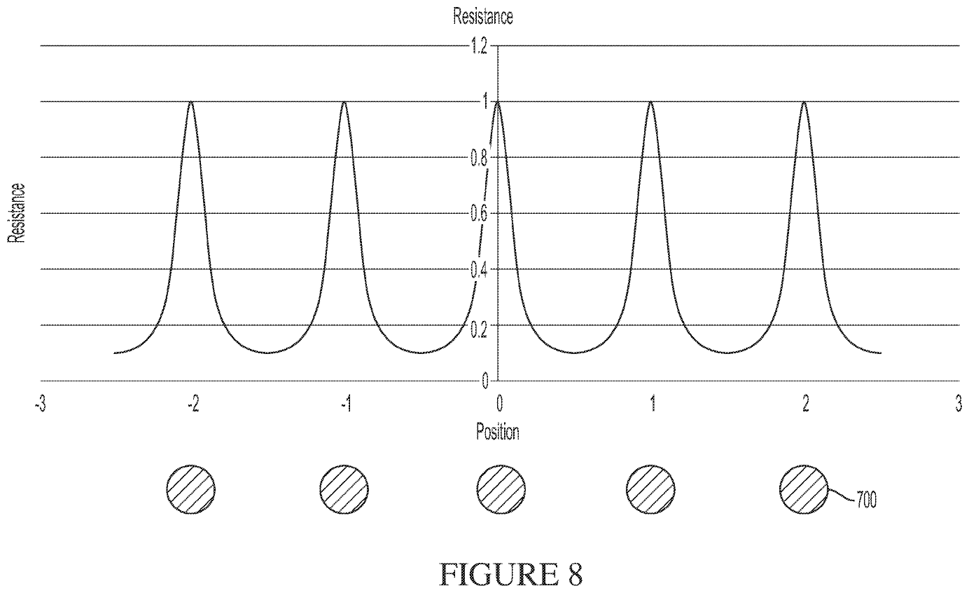

FIG. 8 shows the resistance graph by applying Equation 4 to the sinusoidal flow rate from FIG. 7.

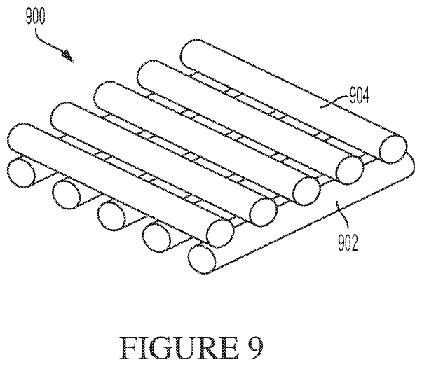

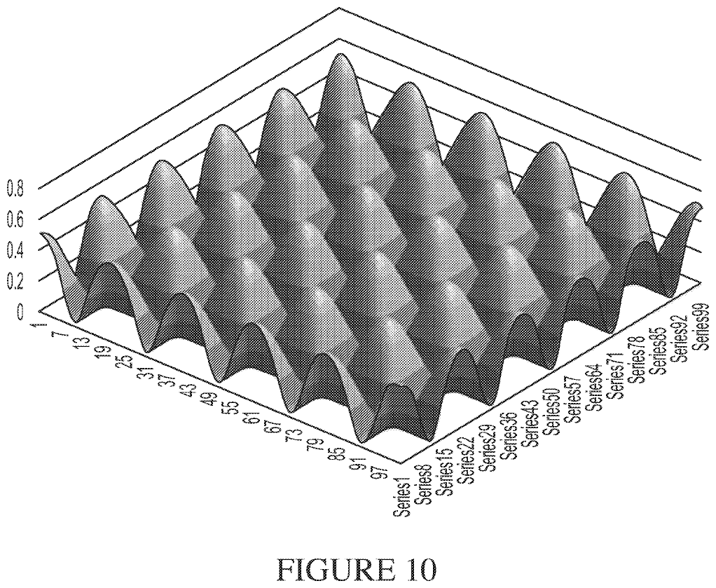

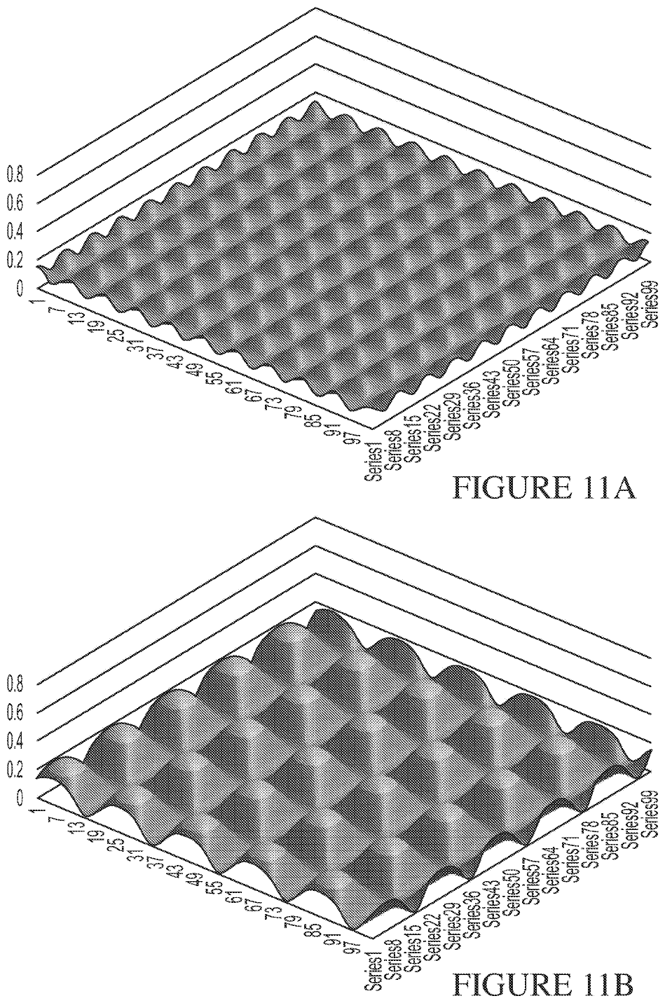

To build on the model, a woven mesh 900 is modeled as two cylinder arrays 902, 904 stacked one over another and running orthogonally to each other, as shown in FIG. 9. It is assumed that the resistance at each location is a linear combination of the resistance from both arrays at that same location. Therefore, the change in flow velocity across the simulated mesh layer can be calculated as a reciprocal function of the final resistance represented at each location, as shown in FIG. 10. Using this approach, the effect of mesh alignment on flow rate distribution can be simulated. For example, when two such meshes are stacked and are completely rotationally aligned (0° rotation relative to each other), there are two boundary conditions: (i) the fibers from the two meshes are fully interlocked (the tight-packed configuration in FIG. 3A and 3B); and (ii) the fibers from the two meshes are fully aligned (the loose-packed configuration in FIG. 4A and 4B). FIGS. 11A and 11B show the modeled flow rate distribution from these tight-packed and loose-packed configurations, respectively. The tight-packed configuration shows the highest resistance to flow and lowest flow rate based on the CFD model result. In the sinusoidal model simulation, the flow rates are lower, as well. Locally, there are small flow velocity oscillations, as shown in FIG. 11A, corresponding to the pattern of the mesh. Over a longer range, the flow rate distribution is generally uniform. In the aligned (loose-packed) configuration, the flow resistance is lowest, and the flow rate is highest based on the CFD model. In the sinusoidal model simulation, the flow rates are higher. In fact, the permeability in this aligned configuration is about ten-times the permeability in the tight-packed configuration. Similarly, there is only local oscillation in flow rate corresponding to the mesh pattern and consistent flow rate over the larger ranges. This means that, in both situations, different regions of the mesh have similar access to flow.

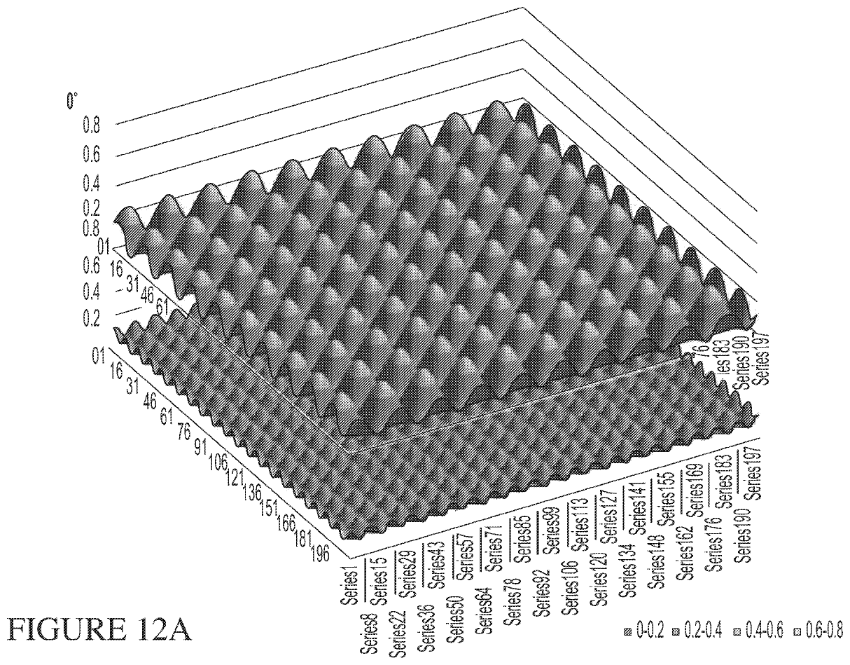

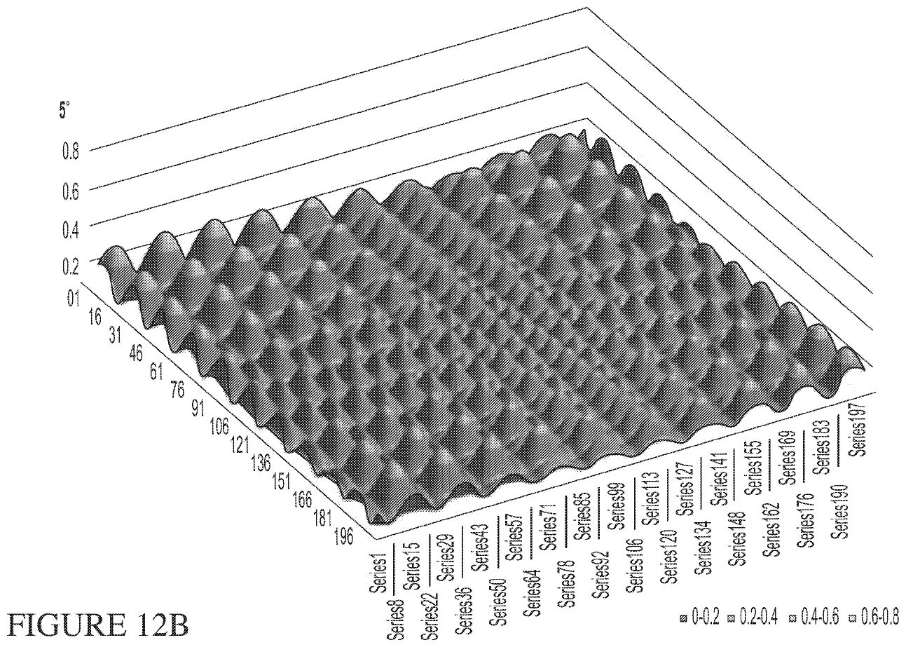

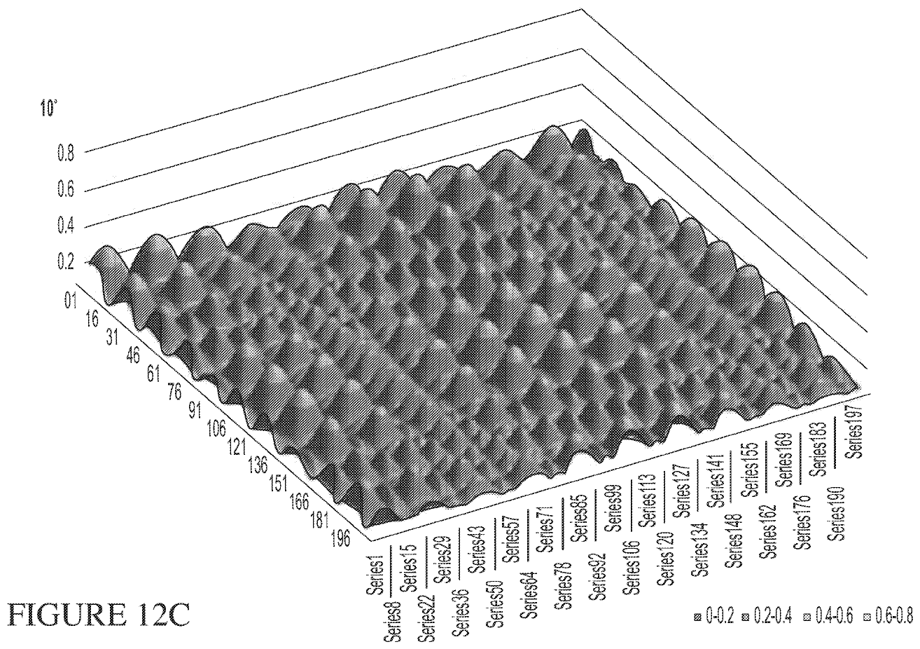

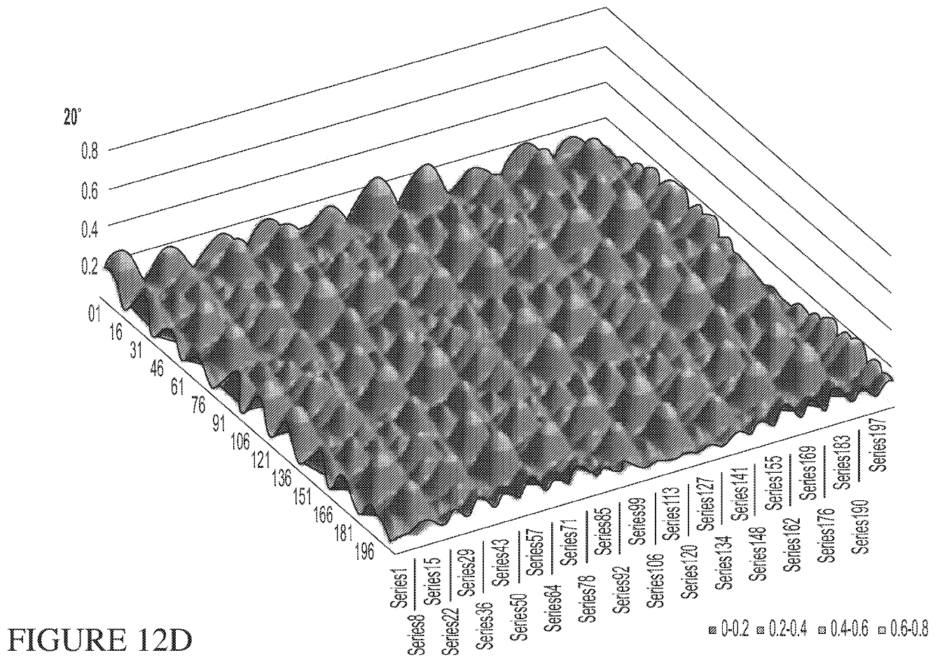

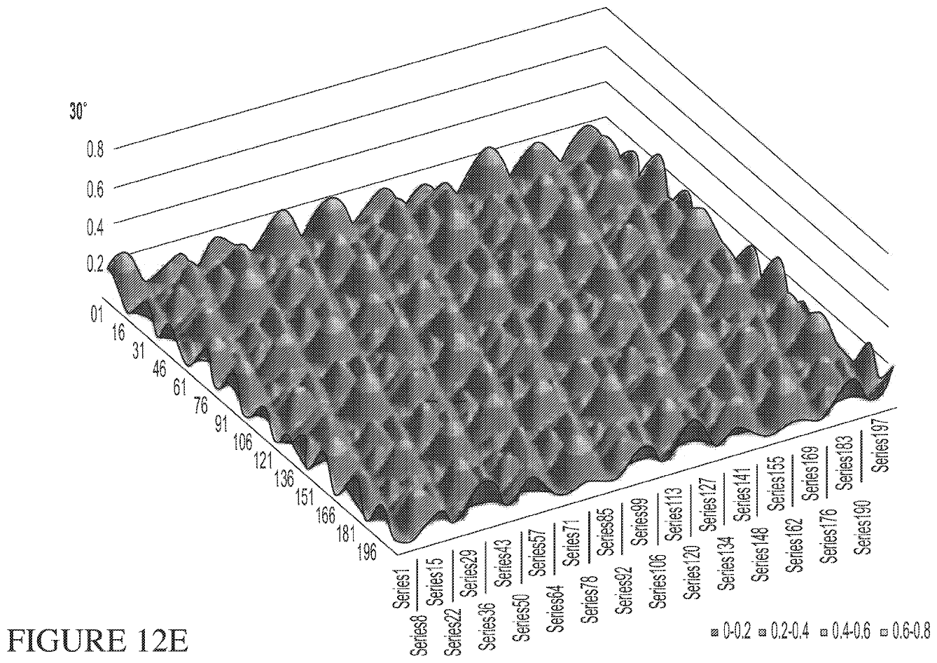

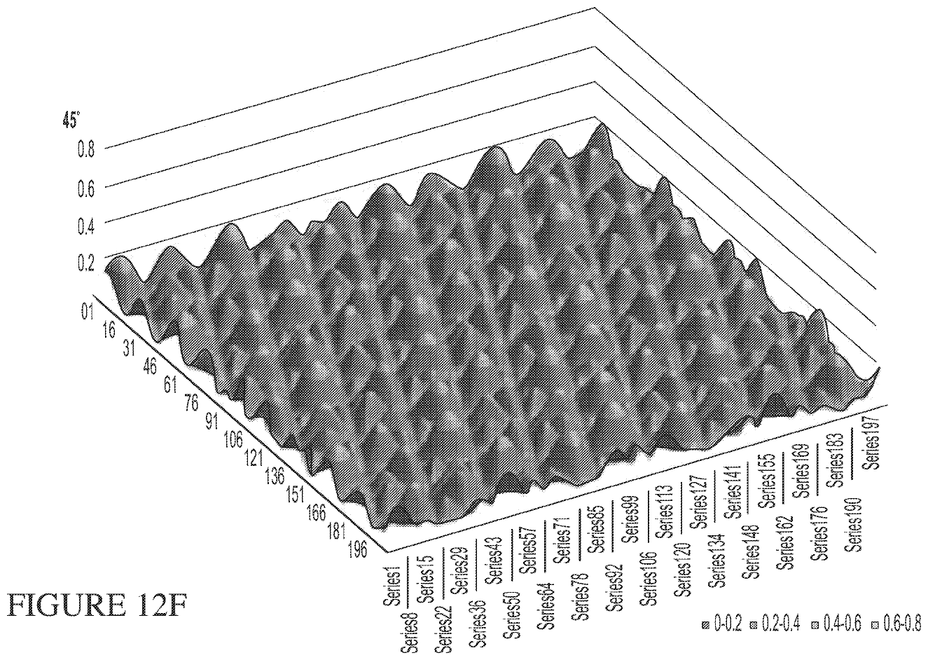

The same approach can now be applied to simulate the flow rate distribution when the two meshes have varying degrees of rotation relative to one another. For example, FIGS. 12A-12F show the simulated flow rate distributions through a stack of two mesh layers with the same physical mesh geometry (i.e., fiber spacing, etc.). FIG. 12A shows the two boundary conditions discussed above, with 0° of relative rotation. FIGS. 12B and 12C show relative rotations of 5° and 10°, respectively, revealing that some areas have significantly higher flow rate than other areas, which agrees with the pattern of opening areas in FIGS. 6C and 6D. FIGS. 12D, 12E, and 12F show relative rotations of 20°, 30°, and 45°, respectively. For alignment of about 20° or higher, the non-uniform flow patterns start to go away, with about 30° to about 45° approaching some level of uniformity. That is, the amount or pattern of flow variation becomes closer in all regions of the mesh stack, although there is still significant nonuniformity.

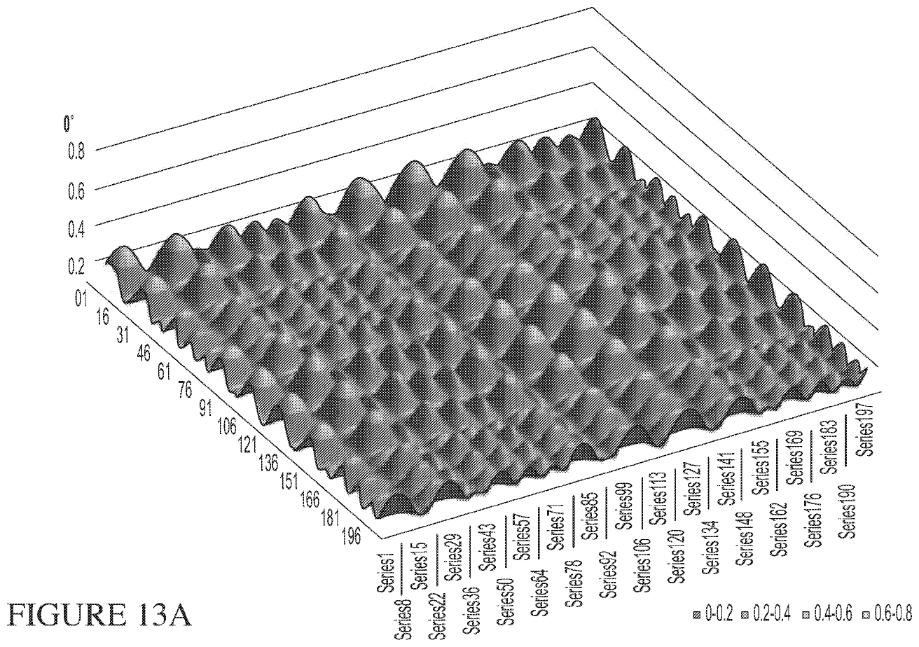

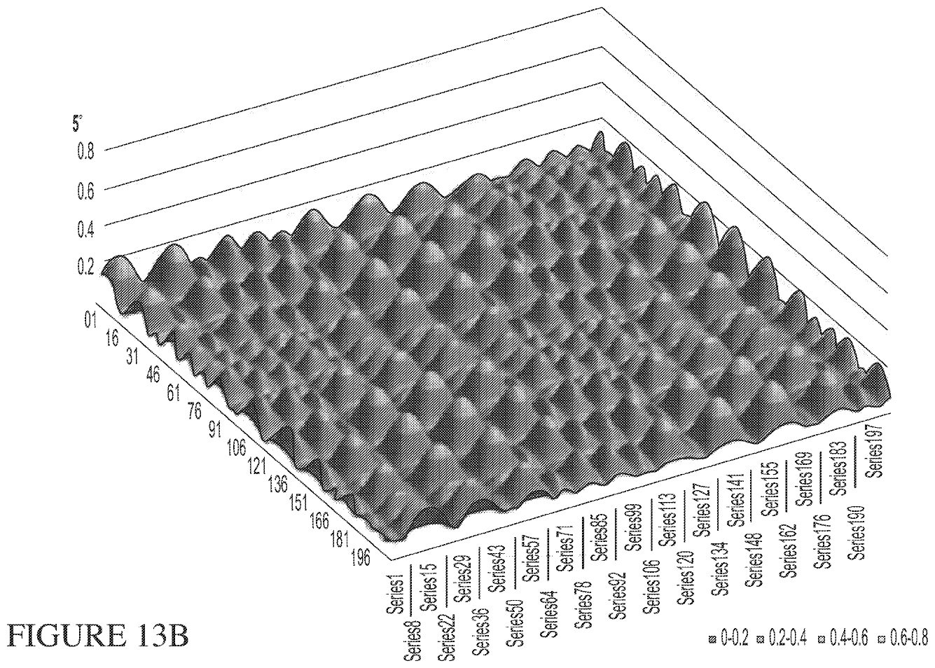

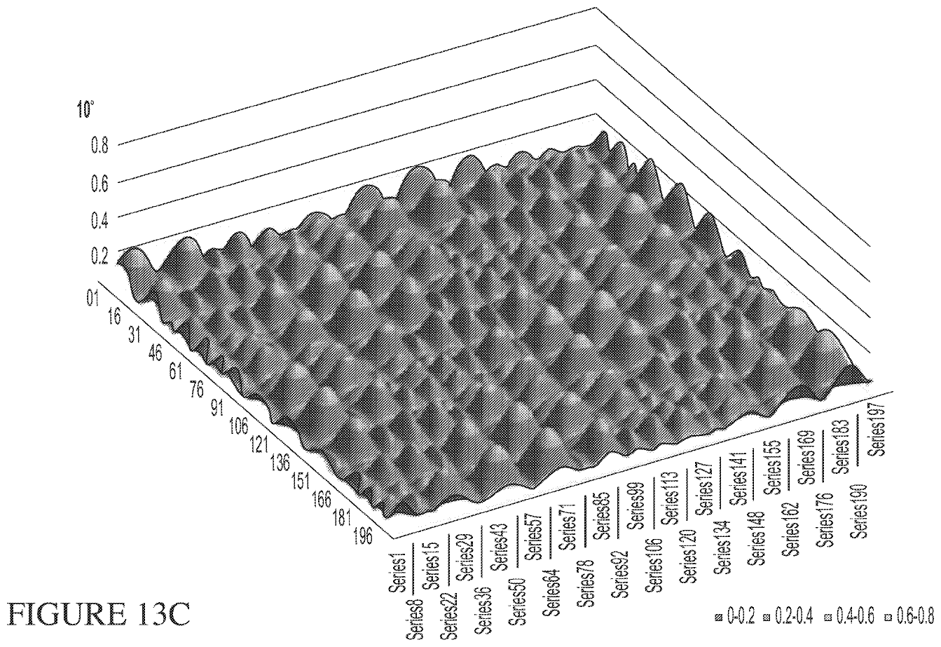

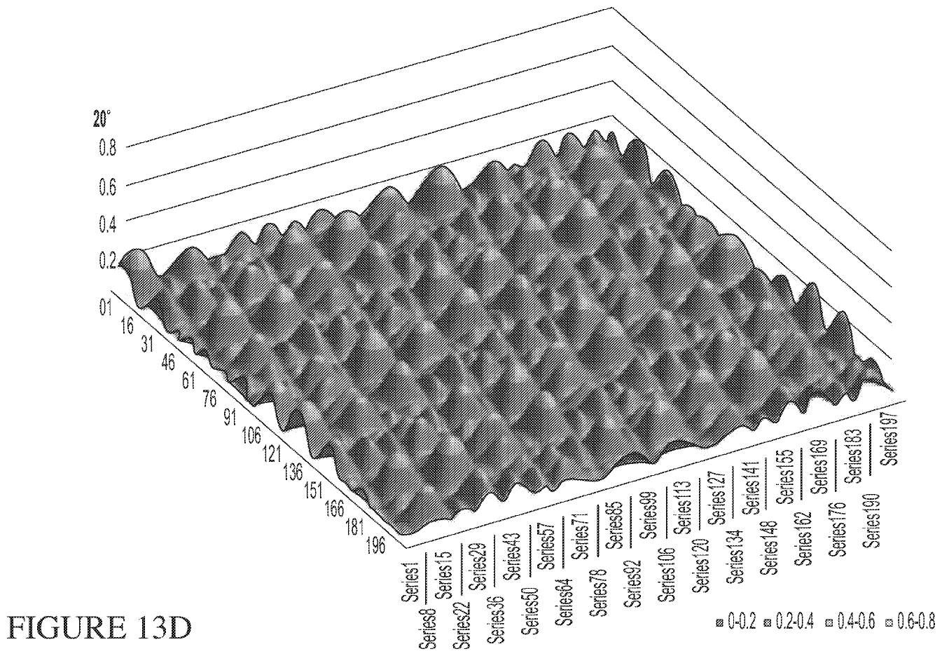

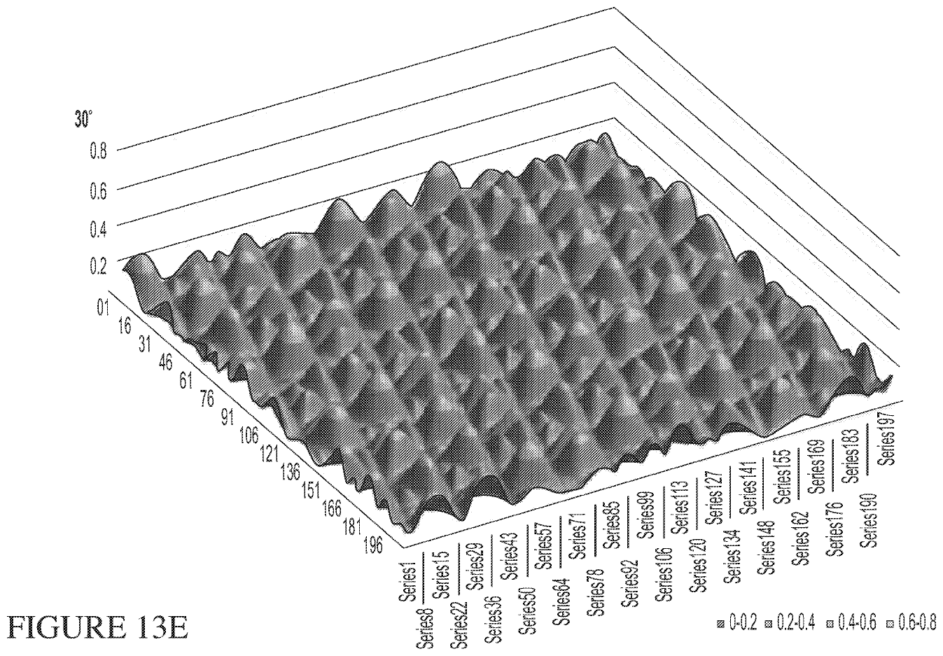

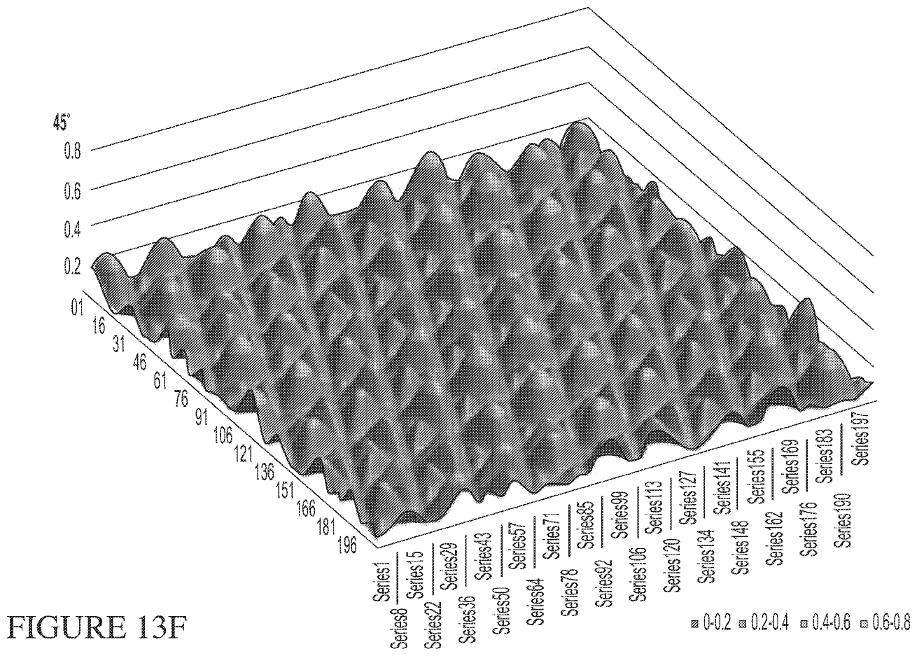

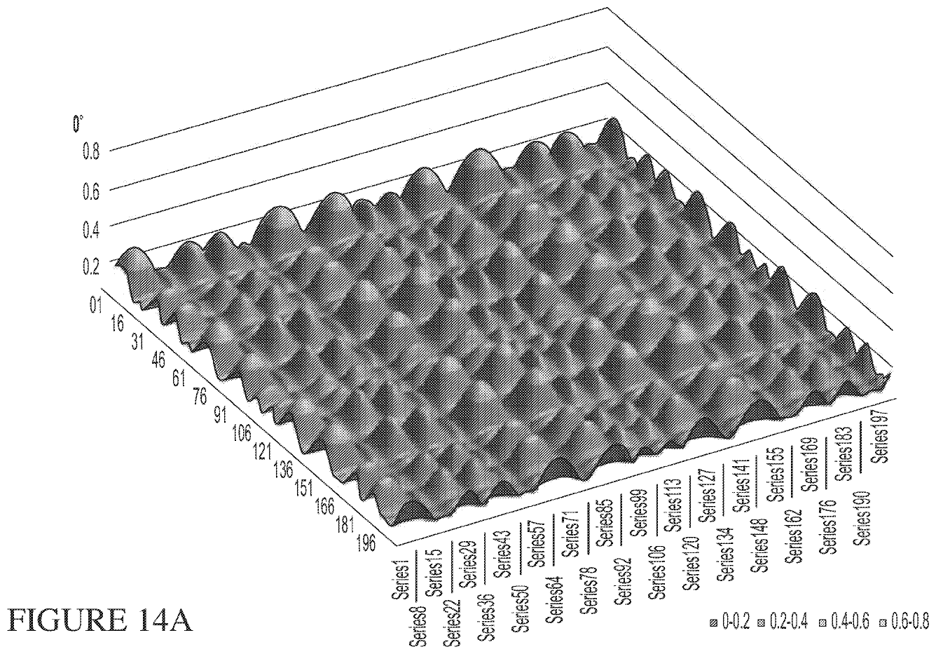

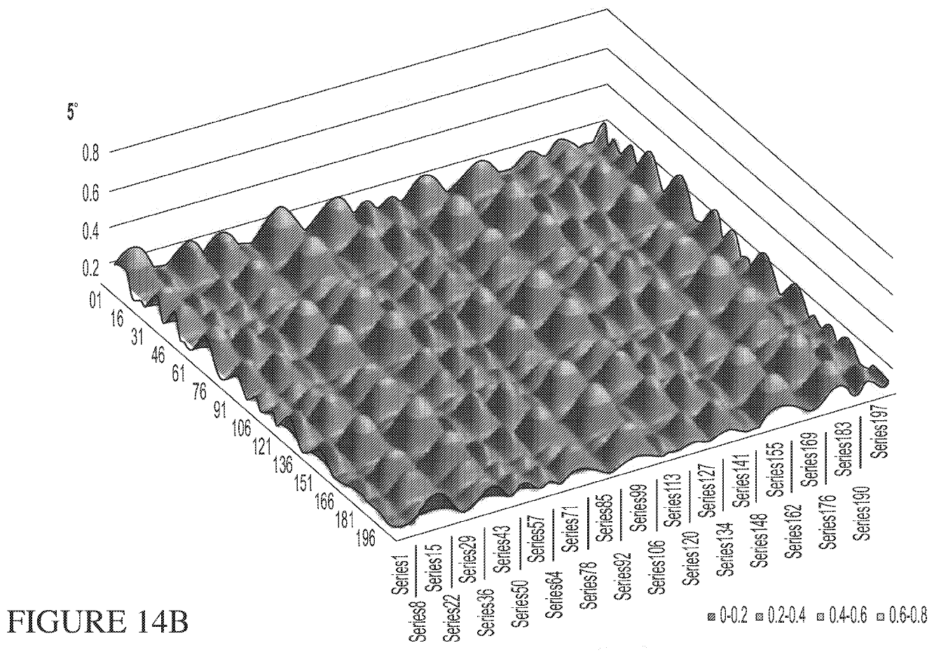

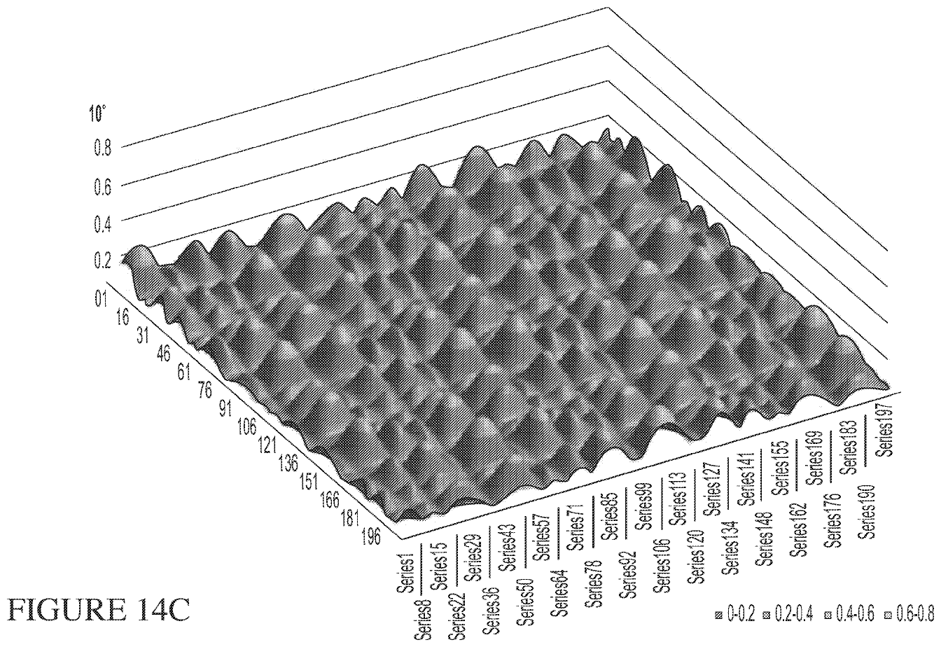

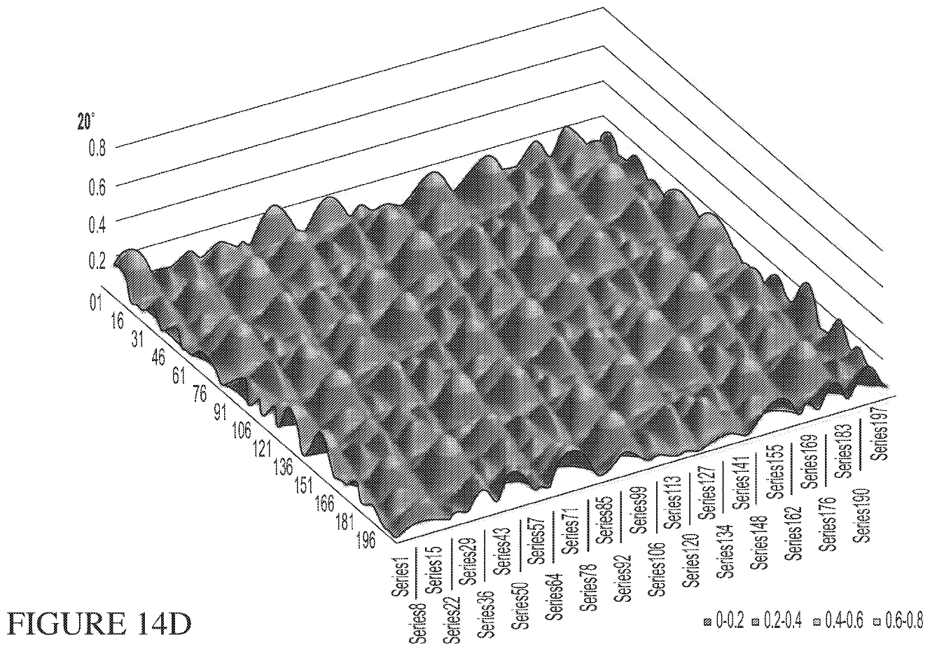

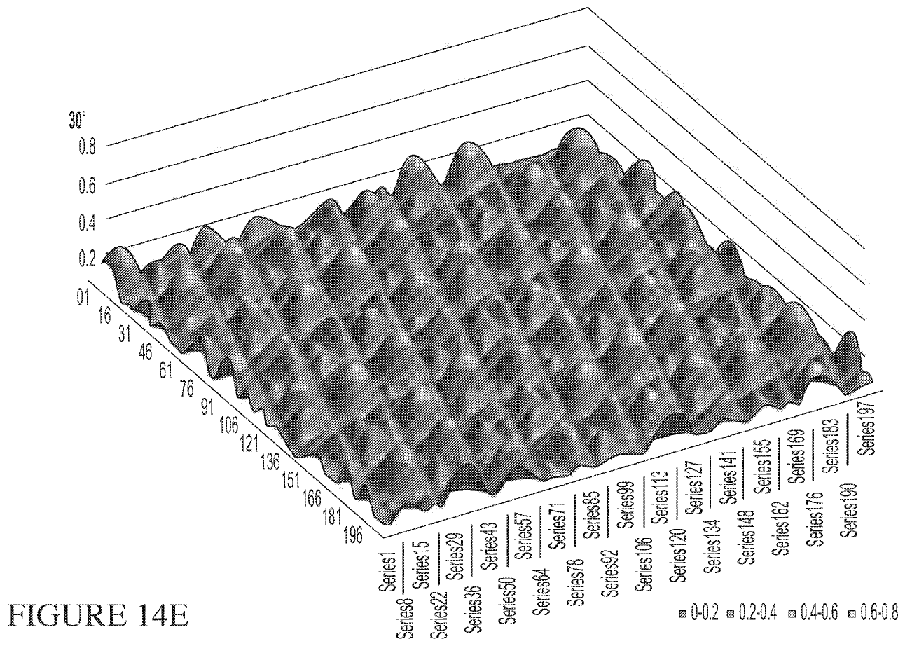

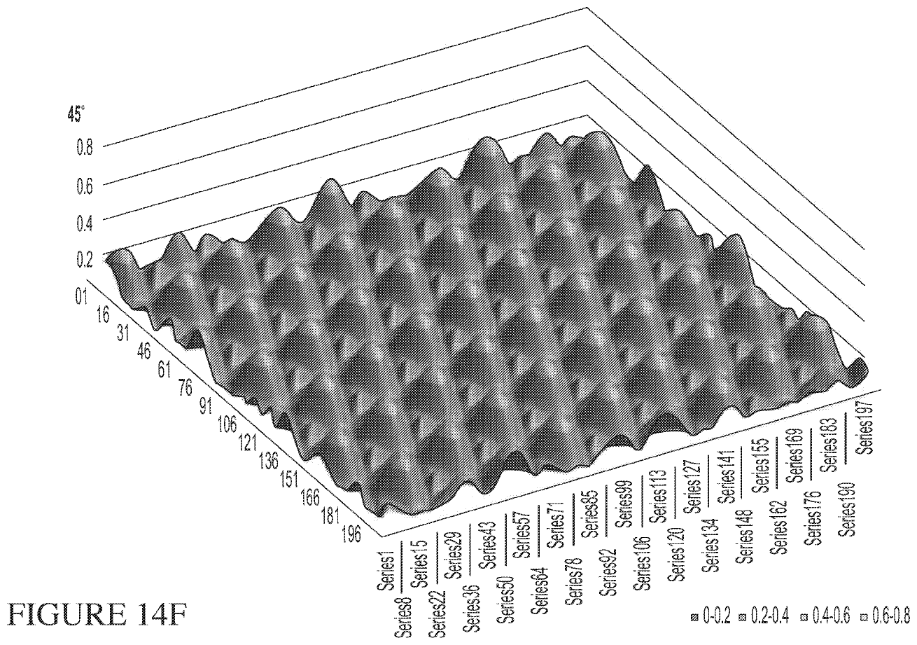

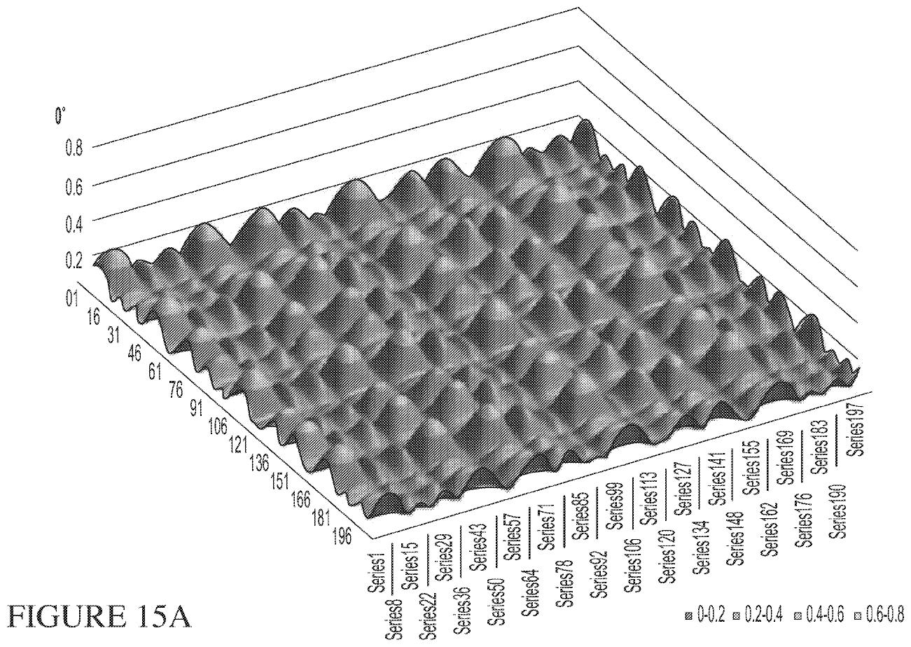

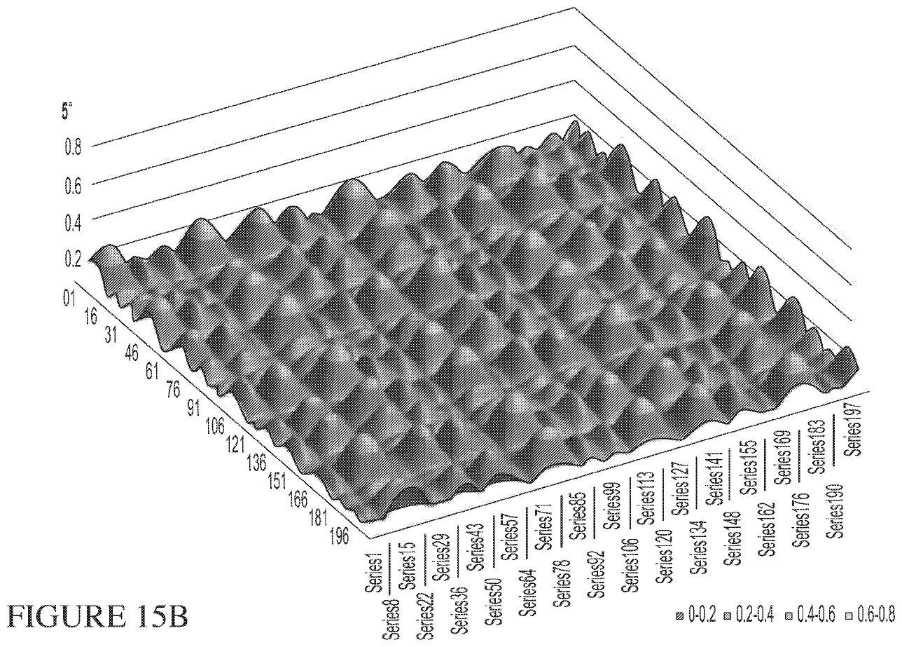

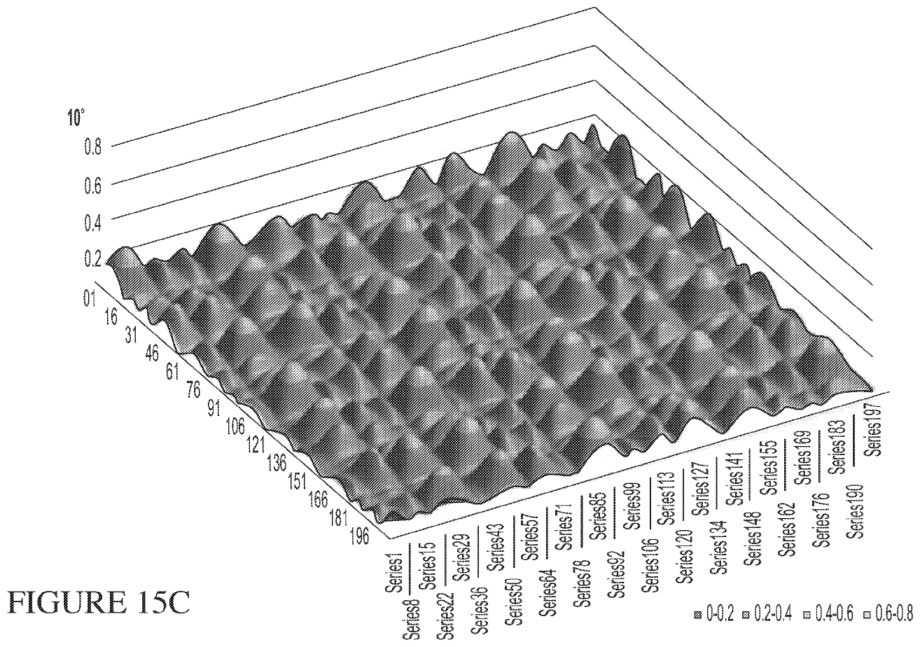

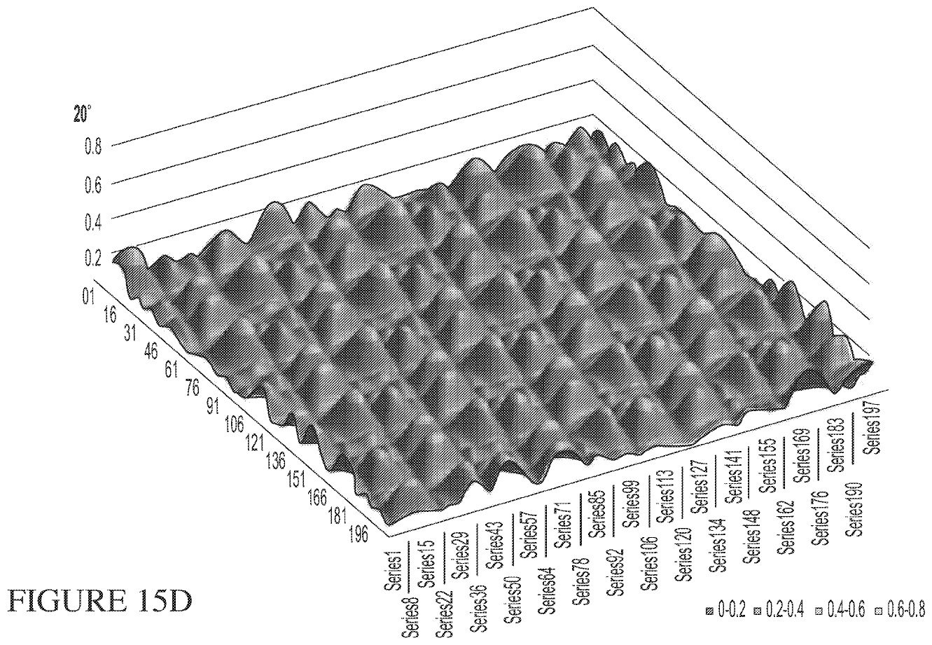

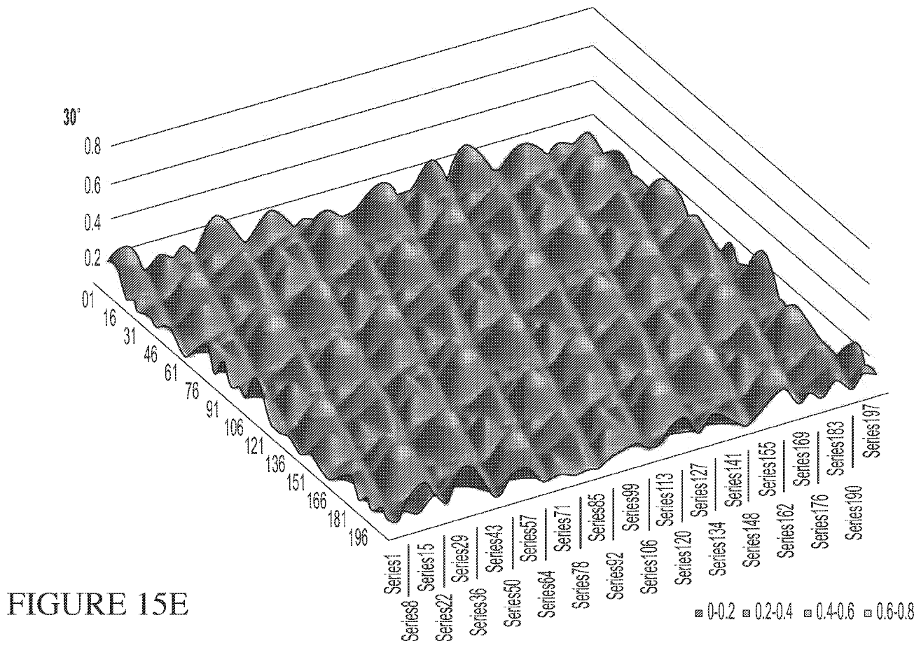

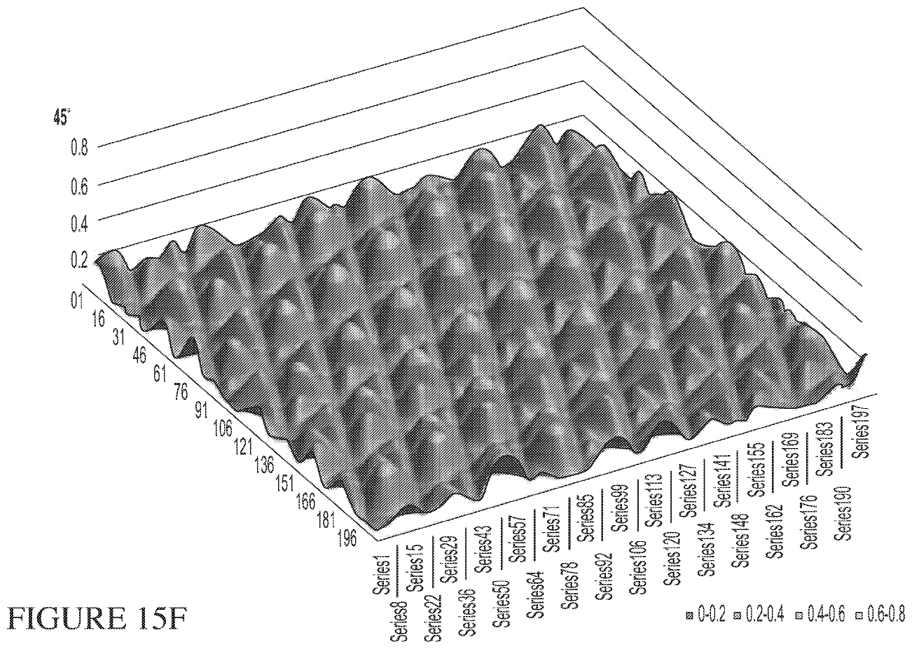

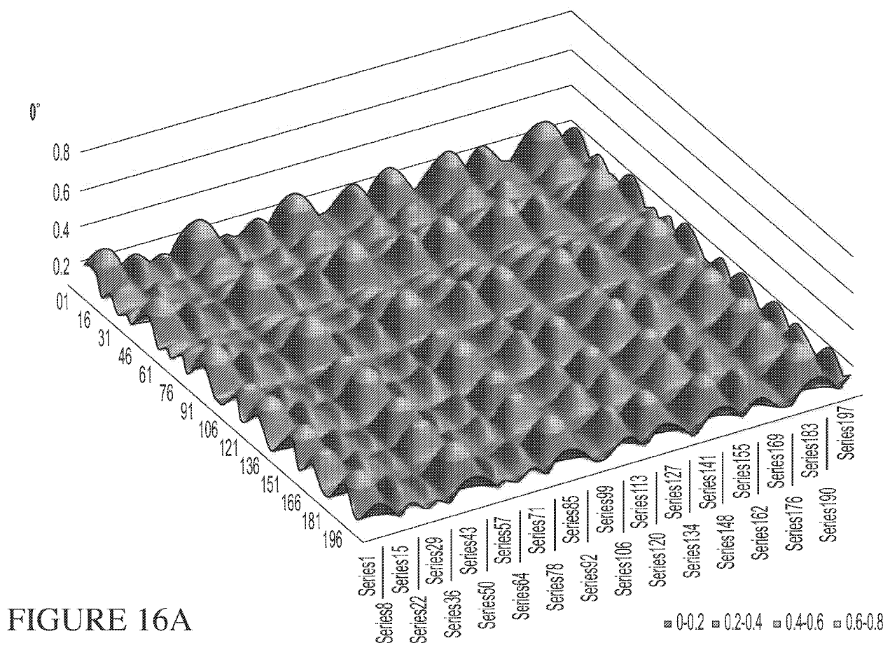

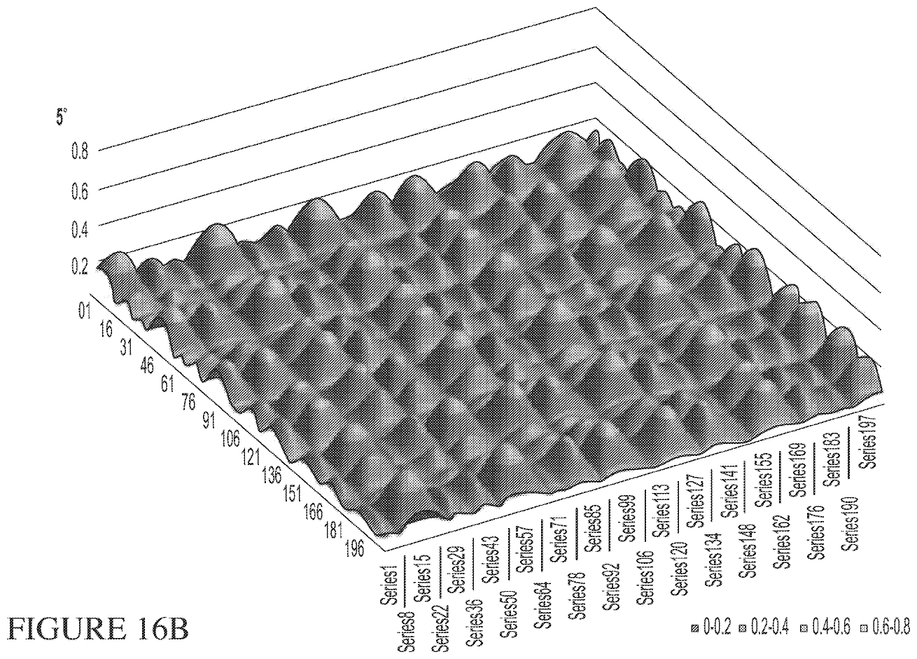

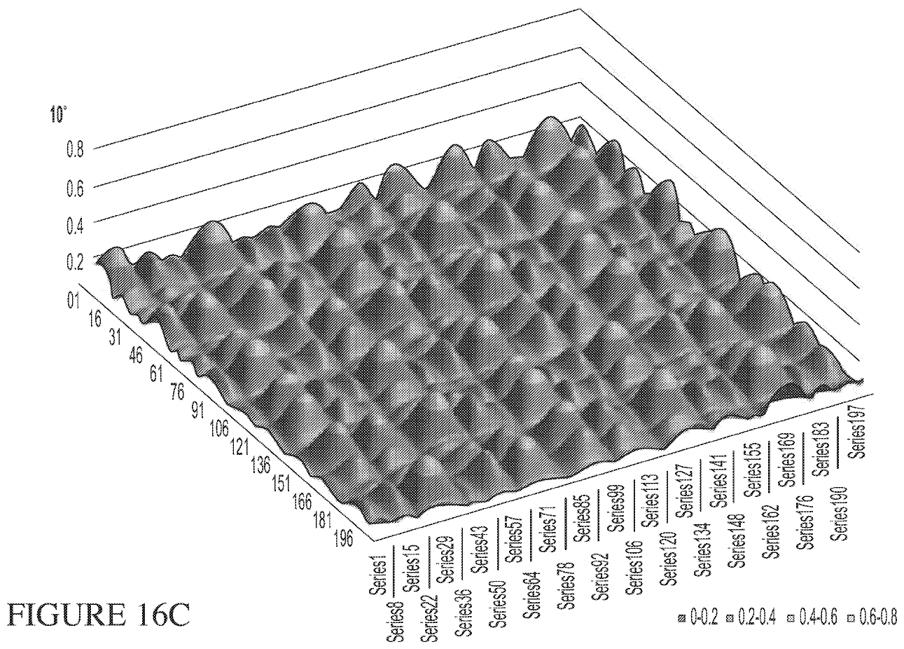

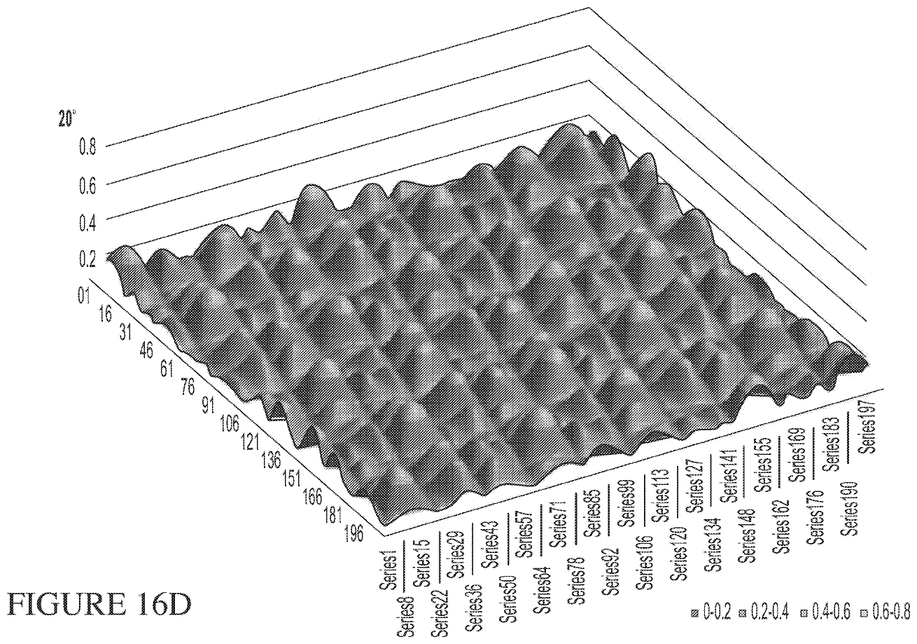

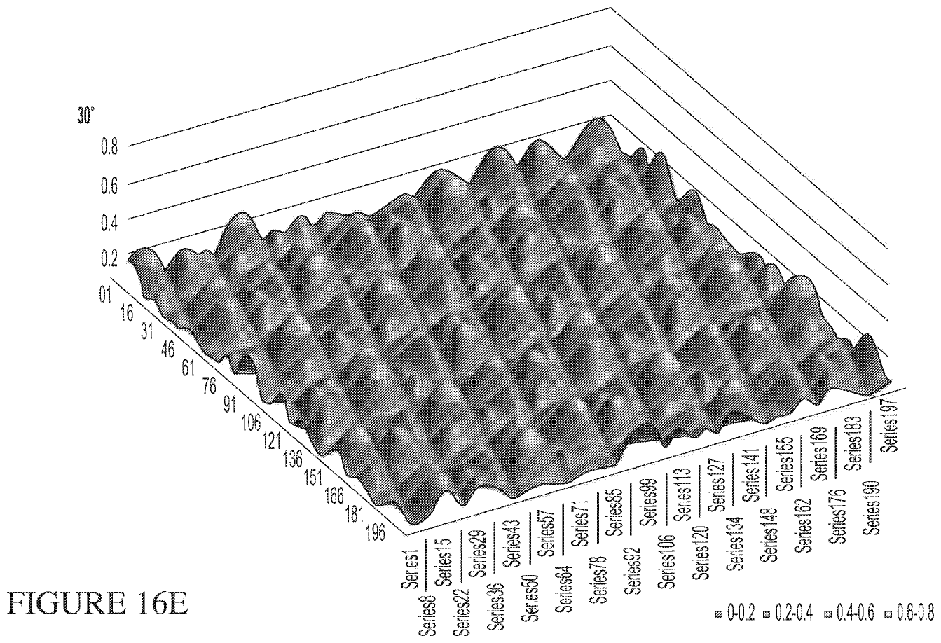

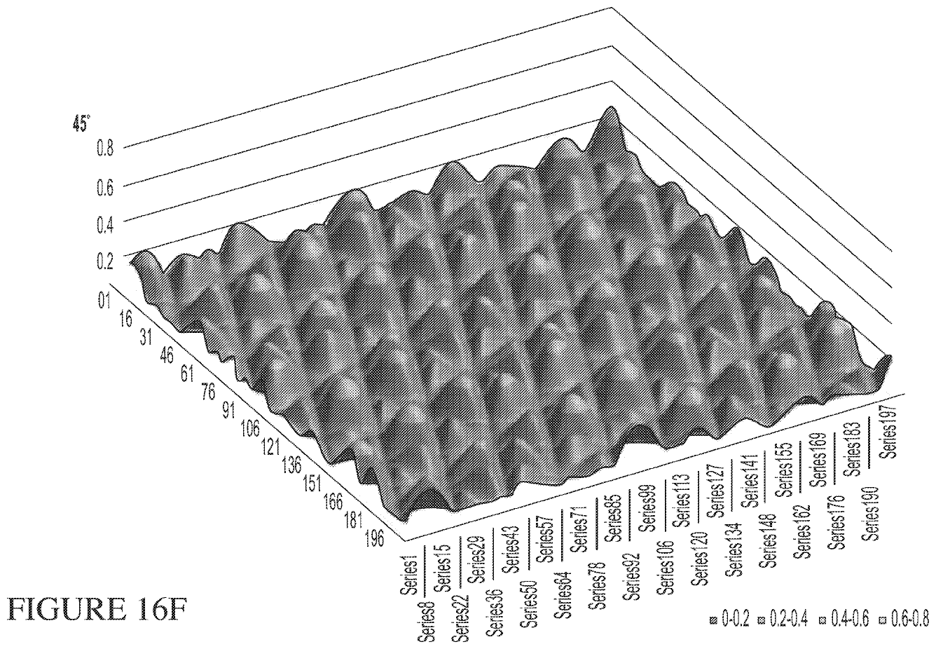

To address this nonuniformity issue, embodiments of this disclose include combining of at least two different types of mesh in a stack. For example, the at least two mesh materials can be stacked in an alternating manner, as discussed herein. To demonstrate the improvement in flow uniformity according to such embodiments, the same simulation can be used as discussed above. For example, FIGS. 13A-16F show the flow patterns when two different types of mesh layers are stacked together. FIGS. 13A-13F show rotational orientations of 0°, 5°, 10°, 20°, 30°, and 45° for two mesh layers with a fiber spacing ratio of 1.2. FIGS. 14A-14F show rotational orientations of 0°, 5°, 10°, 20°, 30°, and 45° for two mesh layers with a fiber spacing ratio of 1.4. FIGS. 15A-15F show rotational orientations of 0°, 5°, 10°, 20°, 30°, and 45° for two mesh layers with a fiber spacing ratio of 1.6. And FIGS. 16A-16F show rotational orientations of 0°, 5°, 10°, 20°, 30°, and 45° for two mesh layers with a fiber spacing ratio of 1.8.

When the fiber spacing ratio between the two mesh layers is 1.2, as in FIGS. 13A-13F, the nonuniformity can still be observed when the meshes are aligned at lower angles, such as 5° or 10°, but the nonuniformity again decreases somewhat at larger degrees of rotation. When the fiber spacing ratio goes to 1.4 (FIGS. 14A-14F) or larger, particularly 1.6 (FIGS. 15A-15F) or larger (e.g., 1.8 in FIGS. 16A-16F), there are almost no discernable areas showing significantly higher flow rate than other areas. This means that using hybrid fixed beds with mesh materials have fiber spacing ratios of about 1.4 or larger should effectively eliminate the impact of alignment to flow uniformity. This will make the control of the fixed bed packing process for a fixed bed reactor much easier during manufacturing, shipping, and end use.

The bioreactor vessel optionally includes one or more outlets capable of being attached to inlet and/or outlet means. Through the one or more outlets, liquid, media, or cells can be supplied to or removed from the chamber. A single port in the vessel may act as both the inlet and outlet, or multiple ports may be provided for dedicated inlets and outlets.

The fixed bed cell culture matrix of embodiments of this disclosure can consist of woven cell culture mesh substrate(s) without any other form of cell culture substrate disposed in or interspersed with the cell culture matrix. That is, the woven cell culture mesh substrate of embodiments of this disclosure are effective cell culture substrates without requiring the type of irregular, non-woven substrates used in existing solution or without any separate and distinct structures or spacers used between layers (e.g., such as those used to create fluid flow channels between layers of substrate). This enables cell culture systems of simplified design and construction, while providing a high-density cell culture substrate with the other advantages discussed herein related to flow uniformity, harvestability, etc.

As discussed herein, the cell culture substrates and bioreactor systems provided offer numerous advantages. For example, the embodiments of this disclosure can support the production of any of a number of viral vectors, such as AAV (all serotypes) and lentivirus, and can be applied toward in vivo and ex vivo gene therapy applications. The uniform cell seeding and distribution maximizes viral vector yield per vessel, and the designs enable harvesting of viable cells, which can be useful for seed trains consisting of multiple expansion periods using the same platform. In addition, the embodiments herein are scalable from process development scale to production scale, which ultimately saves development time and cost. The methods and systems disclosed herein also allow for automation and control of the cell culture process to maximize vector yield and improve reproducibility. Finally, the number of vessels needed to reach production-level scales of viral vectors (e.g., 10¹⁶ to 10¹⁸ AAV VG per batch) can be greatly reduced compared to other cell culture solutions.

Embodiments are not limited to the vessel rotation about a central longitudinal axis. For example, the vessel may rotate about an axis that is not centrally located with respect to the vessel. In addition, the axis of rotation may be a horizonal or vertical axis.

### Definitions

“Wholly synthetic” or “fully synthetic” refers to a cell culture article, such as a microcarrier or surface of a culture vessel, that is composed entirely of synthetic source materials and is devoid of any animal derived or animal sourced materials. The disclosed wholly synthetic cell culture article eliminates the risk of xenogeneic contamination.

“Include,” “includes,” or like terms means encompassing but not limited to, that is, inclusive and not exclusive.

“Users” refers to those who use the systems, methods, articles, or kits disclosed herein, and include those who are culturing cells for harvesting of cells or cell products, or those who are using cells or cell products cultured and/or harvested according to embodiments herein.

“About” modifying, for example, the quantity of an ingredient in a composition, concentrations, volumes, process temperature, process time, yields, flow rates, pressures, viscosities, and like values, and ranges thereof, or a dimension of a component, and like values, and ranges thereof, employed in describing the embodiments of the disclosure, refers to variation in the numerical quantity that can occur, for example: through typical measuring and handling procedures used for preparing materials, compositions, composites, concentrates, component parts, articles of manufacture, or use formulations; through inadvertent error in these procedures; through differences in the manufacture, source, or purity of starting materials or ingredients used to carry out the methods; and like considerations. The term “about” also encompasses amounts that differ due to aging of a composition or formulation with a particular initial concentration or mixture, and amounts that differ due to mixing or processing a composition or formulation with a particular initial concentration or mixture.

“Optional” or “optionally” means that the subsequently described event or circumstance can or cannot occur, and that the description includes instances where the event or circumstance occurs and instances where it does not.

The indefinite article “a” or “an” and its corresponding definite article “the” as used herein means at least one, or one or more, unless specified otherwise.

Abbreviations, which are well known to one of ordinary skill in the art, may be used (e.g., “h” or “hrs” for hour or hours, “g” or “gm” for gram(s), “mL” for milliliters, and “rt” for room temperature, “nm” for nanometers, and like abbreviations).

Specific and preferred values disclosed for components, ingredients, additives, dimensions, conditions, and like aspects, and ranges thereof, are for illustration only; they do not exclude other defined values or other values within defined ranges. The systems, kits, and methods of the disclosure can include any value or any combination of the values, specific values, more specific values, and preferred values described herein, including explicit or implicit intermediate values and ranges.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is in no way intended that any particular order be inferred.

It will be apparent to those skilled in the art that various modifications and variations can be made without departing from the spirit or scope of the disclosed embodiments. Since modifications, combinations, sub-combinations and variations of the disclosed embodiments incorporating the spirit and substance of the embodiments may occur to persons skilled in the art, the disclosed embodiments should be construed to include everything within the scope of the appended claims and their equivalents.

1. A cell culture matrix for culturing cells in a fixed bed reactor, the cell culture matrix comprising:
a plurality of substrate layers in a stacked arrangement of parallel layers, each layer of the plurality of substrate layers comprising a substrate material with an ordered and regular array of openings passing through the layer, the openings being separated by the substrate material having a physical structure that is substantially regular and uniform and that is configured for growing cell thereon,
wherein the plurality of substrate layers comprises a first substrate material and a second substrate material that is different from the first substrate material in at least one physical dimension, the first substrate material and the second substrate material comprising separate layers of the plurality of substrate layers, and
wherein the physical structure comprises a plurality of fibers.

2. The cell culture matrix of claim 1, wherein the at least one physical dimension is at least one of a diameter of the openings, a thickness of the physical structure, a pattern of the physical structure, and a spacing of the physical structure on either side of an opening.

3-6. (canceled)

7. The cell culture matrix of claim 1, wherein the at least one physical dimension is a fiber spacing between two neighboring fibers of the plurality of fibers.

8. The cell culture matrix of claim 7, wherein a ratio of the fiber spacing of the first substrate material to the fiber spacing of the second substrate material is greater than 1.0, about 1.2 or greater, about 1.4 or greater, about 1.6 or greater, about 1.8 or greater, or from about 1.4 to about 1.8.

9. The cell culture matrix of claim 1, wherein a degree of relative rotation between the first substrate material and the second substrate material is random.

10. The cell culture matrix of claim 1, wherein a variation in flow rate of fluid flowing through the cell culture matrix is uniform across a width of the cell culture matrix, the width being in a direction perpendicular to a direction of fluid flow.

11. The cell culture matrix of claim 1, wherein the first substrate material and the second substrate material are stacked as alternating layers of the plurality of substrate layers.

12. (canceled)

13. (canceled)

14. The cell culture matrix of claim 1, wherein the plurality of fibers comprises a first fiber with a first fiber diameter from about 10 μm to about 1000 μm, from about 15 μm to about 600 μm, from about 20 μm to about 400 μm, from about 30 μm to about 325 μm, from about 15 μm to about 200 μm, or from about 150 μm to about 275 μm.

15. The cell culture matrix of claim 1, wherein the openings comprise a diameter of rom about 20 μm to about 1000 μm, from about 40 μm to about 900 μm, from about 50 μm to about 300 μm, or from about 225 μm to about 800 μm.

16-18. (canceled)

19. A cell culture matrix for culturing cells in a fixed bed reactor, the cell culture matrix comprising:
a plurality of substrate layers in a stacked arrangement of parallel layers, each layer of the plurality of substrate layers comprising a plurality of fibers configured for culturing cells thereon, and an ordered and regular array of openings defined by the plurality of fibers and passing through the layer,
wherein each layer of the plurality of substrate layers is rotated about a center of the layer relative to an immediately neighboring layer in the stack.

20. The cell culture matrix of claim 19, wherein a degree of the rotation is about 20° or greater, about 20° to about 45°, about 30° to about 45°, or about 45°.

21. The cell culture matrix of claim 19, wherein at least a portion of the plurality of substrates are not separated by a spacer material or barrier, or are in physical contact with each other.

22. The cell culture matrix of claim 19, wherein the plurality of substrate layers comprises at least one of polystyrene, polyethylene terephthalate, polycarbonate, polyvinylpyrrolidone, polybutadiene, polyvinylchloride, polyethylene oxide, polypyrroles, and polypropylene oxide.

23. The cell culture matrix of claim 19, wherein the plurality of fibers comprises a first fiber with a first fiber diameter from about 10 μm to about 1000 μm, from about 15 μm to about 600 μm, from about 20 μm to about 400 μm, from about 30 μm to about 325 μm, from about 15 μm to about 200 μm, or from about 150 μm to about 275 μm.

24. The cell culture matrix of claim 19 , wherein the openings comprise a diameter of rom about 20 μm to about 1000 μm, from about 40 μm to about 900 μm, from about 50 μm to about 300 μm, or from about 225 μm to about 800 μm.

25. The cell culture matrix of claim 19, wherein the plurality of fibers are interwoven.

26. The cell culture matrix of claim 19, wherein each layer of the plurality of substrate layers is rotated about the center of the layer relative to an immediately neighboring layer in the stack such that an orientation of fibers in one layer of the plurality of substrate layers is different from an orientation of fibers in an immediately adjacent layer of the plurality of substrate layers.

27. The cell culture matrix of claim 26, wherein a difference in the orientation of fibers in the one layer and the immediately adjacent layer is:
about 5° or more, about 10° or more, about 15° or more, about 20° or more, about 25° or more, about 30° or more, or about 40° or more, and
less than about 90°, about 85° or less, about 80° or less, about 75° or less, about 65° or less, about 60° or less, or about 50° or less.

28. The cell culture matrix of claim 27, wherein the difference in the orientation is about 45°.

29. The cell culture matrix of claim 27, wherein the difference in the orientation is from about 40° to about 50°.
